(19) United States
(12) Patent Application Publication
(10) Pub. No.: US 2023/0375544 A1
(43) Pub. Date: Nov. 23, 2023

(54) Methods for Determining a Level of Cell-Bound Complement Activation Product

(71) Applicant: Allegheny Singer Research Institute, Pittsburgh, PA (US)

(72) Inventors: Joseph M. Ahearn, Wexford, PA (US); Chau-Ching Liu, Pittsburgh, PA (US)

(21) Appl. No.: 18/246,792

(22) PCT Filed: Sep. 28, 2021
(86) PCT No.: PCT/US21/52437

§ 371 Date: Mar. 27, 2023

Related U.S. Application Data
(60) Provisional application No. 63/248,962, filed on Sep. 27, 2021.
(60) Provisional application No. 63/084,432, filed on Sep. 28, 2020.

(51) Int. Cl. G01N 33/564; C07K 16/18

(52) U.S. Cl. CPC G01N 33/564; C07K 16/18; G01N 2333/4716; G01N 2800/104; C07K 2317/31

(57) ABSTRACT

This disclosure provides a novel method for detecting one or more cell fragment-bound complement activation products (CFB-CAPs) using a capillary flow system. The method eliminates the need for fresh, live cellular samples and detection by flow cytometric methods. The method as disclosed has a wide variety of applications, including diagnosing or monitoring lupus or pre-lupus and other diseases or disorders (e.g., autoimmune or inflammatory diseases or disorders).

### CROSS-REFERENCE TO RELATED APPLICATIONS

This application claims priority to and is a national phase filing of International Patent Application No. PCT/US2021/52437 filed Sep. 28, 2021, which claims priority under 35 U.S.C. § 119(e) to U.S. Provisional Patent Application No. 63/084,432, filed Sep. 28, 2020, and U.S. Provisional Patent Application No. 63/248,962 filed Sep. 27, 2021. The foregoing applications are incorporated by reference herein in their entirety.

### TECHNICAL FIELD

This document relates to methods for determining a level of a cell fragment-bound complement activation product (CFB-CAP), and more specifically to methods for determining a level of a CFB-CAP in a sample from a patient using a capillary flow system.

### BACKGROUND

Cell-bound complement activation products (CB-CAP) have been validated for diagnosis, monitoring, and stratification of lupus and pre-lupus. In addition to their roles as lupus biomarkers, cell-bound complement activation products have been shown to confer functional abnormalities to circulating cells such as erythrocytes and T lymphocytes, suggesting a role in lupus pathogenesis. The profiling of cell-bound complement activation products serves as diagnostic biomarkers for identifying lupus or pre-lupus in a patient. However, the existing assays for detecting cell-bound complement activation products require fresh, live, and intact cellular samples and detection by flow cytometric methods, making them costly and less convenient to use.

### SUMMARY

In one aspect, this disclosure provides a method of determining a level of a complement activation product in a patient, such as a complement activation product attached to a cell fragment (referred to as a cell fragment-bound complement activation product (“CFB-CAP”)). The method comprises: (i) contacting a sample comprising one or more analytes with a detection agent, wherein the one or more analytes comprise a cell fragment and a CFB-CAP, and wherein the detection agent comprises a detection antibody (such as an anti-C4d antibody) that specifically binds to the CFB-CAP and facilitates detection of the CFB-CAP in at least one of the one or more analytes; (ii) immobilizing the at least one of the one or more analytes in a fluid path; and (iii) determining a level of the CFB-CAP in the at least one of the one or more analytes at one or more locations in the fluid path by determining a level of binding of the detection antibody to the CFB-CAP. In some embodiments, the sample comprises a blood sample.

In some embodiments, the CFB-CAP is attached to at least a fragment (such as a cell fragment) of erythrocytes, lymphocytes, reticulocytes, platelets, granulocytes, monocytes, eosinophils, or basophils.

In some embodiments, the sample comprises at least one entity selected from erythrocytes, lymphocytes, reticulocytes, platelets, granulocytes, monocytes, eosinophils, and basophils, and the method comprises lysing the at least one entity in the sample before contacting the sample with the detection agent.

In some embodiments, the sample, after being obtained from the patient, has been left at a temperature equal to or above 4 degrees Celsius for a least a period of time before being contacted with the detection agent. In some embodiments, at least a portion of the entity has been spontaneously lysed. In some embodiments, the method comprises holding the sample at a temperature equal to or above 4 degrees Celsius for at least a period of time before contacting the sample with the detection agent, whereby a least a portion of the entity is spontaneously lysed. In some embodiments, the period of time is at least about 60 minutes.

In some embodiments, the method further comprises fixing the one or more analytes with a fixation reagent.

In some embodiments, the CFB-CAP comprises a cell fragment-bound C4d. In some embodiments, the detection antibody comprises an anti-C4d antibody. In some embodiments, the cell fragment-bound C4d comprises any one of BC4d, TC4d, EC4d, PC4d, RC4d, GC4d, MC4d, and combinations thereof.

In some embodiments, the at least one of the one or more analytes is immobilized in the fluid path by a capture antibody (such as an anti-C4d antibody). In some embodiments, the capture antibody binds to a different epitope on the CFB-CAP from one to which the detection antibody binds or binds to the detection antibody. In some embodiments, the capture antibody is an anti-C4d antibody.

In some embodiments, the capture antibody comprises a tag or a moiety that immobilizes the capture antibody in the fluid path.

In some embodiments, the one or more analytes further comprise an anti-T cell antibody. In some embodiments, the method comprises determining a level of at least one of the CFB-CAP and the anti-T cell antibody in the one or more analytes. In some embodiments, the method comprises determining a level of each of the CFB-CAP and the anti-T cell antibody in the one or more analytes.

In some embodiments, the capture antibody comprises a first capture antibody that binds to the CFB-CAP and a second capture antibody that binds to the anti-T cell antibody. In some embodiments, the first capture antibody and the second capture antibody are configured to immobilize the CFB-CAP and the anti-T cell antibody at at least two separate locations in the fluid path. In some embodiments, the anti-T cell antibody is an anti-T cell autoantibody.

In some embodiments, the method comprises determining a level of each of the CFB-CAP and the anti-T cell antibody in a single fluid path (such as in the same test strip).

In some embodiments, the one or more analytes further comprise a freely circulating complement activation product (FC-CAP). In some embodiments, the method comprises determining a level of each of the CFB-CAP and the FC-CAP in the one or more analytes.

In some embodiments, the capture antibody comprises a first capture antibody that binds to the CFB-CAP and a second capture antibody that binds to the FC-CAP. In some embodiments, the first capture antibody and the second capture antibody are configured to immobilize the CFB-CAP and the FC-CAP at at least two separate locations in the fluid path. In some embodiments, the method comprises determining a level of each of the CFB-CAP and the FC-CAP in a single fluid path (such as in the same test strip).

In some embodiments, the one or more analytes containing the CFB-CAP and the FC-CAP further comprise an anti-T cell antibody. In some embodiments, the method comprises determining a level of at least one of the CFB-CAP, the FC-CAP, and the anti-T cell antibody in the one or more analytes. In some embodiments, the method comprises determining a level of each of the CFB-CAP, the FC-CAP, and the anti-T cell antibody in the one or more analytes. In some embodiments, the capture antibody comprises a first capture antibody that binds to the CFB-CAP, a second capture antibody that binds to the FC-CAP, and a third capture antibody that binds to the anti-T cell antibody, wherein the first capture antibody, the second capture antibody, and the third capture antibody are configured to immobilize the CFB-CAP, the FC-CAP, and the anti-T cell antibody at two or more separate locations (such as three separate locations) in the fluid path.

In some embodiments, the method comprises comprising determining a level of each of the CFB-CAP, the anti-T cell antibody, and the FC-CAP in a single fluid path (such as in the same test strip).

In some embodiments, the detection agent further comprises enzyme substrates or chemiluminescent substrates. In some embodiments, detecting binding of the detection antibody to the CFB-CAP comprises detecting a chemiluminescent signal.

In some embodiments, the detection antibody comprises a label. In some embodiments, the label comprises a nanoparticle label, a fluorescent label, a chemiluminescent label, a radiolabel, or an enzyme.

In some embodiments, the detection antibody or the capture antibody is a bispecific antibody, a trispecific antibody, a single chain Fv (scFv), a monoclonal antibody, a chimeric antibody, a humanized antibody, a recombinant antibody, or a human antibody. In some embodiments, the bispecific antibody comprises a first antigen-binding arm binding to C4d and a second antigen-binding arm binding to any one of CD3, CD4, CD5, CD8, CD45, CD19, CD20, CD21, CD22, CD23, CD25, CD40, CD42b, CD69, CD70, CD79, CD80, CD85, CD86, CD137, CD138, CD252, and CD268. In some embodiments, the chimeric antibody comprises a human Fc domain and a murine variable region.

In some embodiments, the fluid path comprises a test strip that comprises a wicking material. In some embodiments, the step of immobilizing is performed before the step of contacting.

In another aspect, this disclosure provides a method of identifying lupus or pre-lupus in a patient. The method comprises: (i) obtaining a sample (such as a blood sample) from the patient; (ii) determining a level of the CFB-CAP in the sample by a method described above; (iii) comparing the determined level of the CFB-CAP with a control level and determining whether the determined level is elevated as compared to the control level; and (iv) determining that the patient has lupus or an increased risk of developing lupus if the determined level of the CFB-CAP is elevated as compared to the control level.

In some embodiments, the method further comprises: (a) determining a level of an anti-T cell antibody contained in the blood sample by the method as described herein; (b) comparing the determined level of the anti-T cell antibody with a second control level and determining whether the determined level of the anti-T cell antibody is elevated as compared to the second control level; and (c) determining that the patient has lupus or an increased risk of developing lupus if the determined level of the CFB-CAP and the determined level of the anti-T cell antibody are elevated as compared to the control level and the second control level, respectively.

In yet another aspect, this disclosure provides a method of identifying a disease or disorder in an individual. The method comprises: (a) obtaining a bodily fluid sample from the patient; (b) determining a level of the CFB-CAP contained in the bodily fluid sample by the method described above; (c) comparing the determined level of the CFB-CAP with a control level and determining whether the determined level is elevated as compared to the control level; and (d) determining that the patient has the disease or disorder if the determined level of the CFB-CAP is elevated as compared to the control level. In some embodiments, the disease or disorder is an autoimmune disease or inflammation. In some embodiments, the disease or disorder is systemic lupus erythematosus.

In some embodiments, the method further comprises: (a) determining a level of an anti-T cell antibody contained in the bodily fluid sample by the method as described herein; (b) comparing the determined level of the anti-T cell antibody with a second control level and determining whether the determined level of the anti-T cell antibody is elevated as compared to the second control level; and (c) determining that the patient has the disease or disorder if the determined level of the CFB-CAP and the determined level of the anti-T cell antibody are elevated as compared to the control level and the second control level, respectively.

In yet another aspect, this disclosure provides a method of monitoring progression of a disease or disorder in an individual. The method comprises: (i) obtaining a bodily fluid sample from the patient; (ii) determining a level of the CFB-CAP contained in the bodily fluid sample by the method as described herein; (iii) comparing the determined level of the CFB-CAP with a control level and determining whether the determined level is elevated or decreased as compared to the control level; and (iv) determining that (a) the patient has progression of the disease or disorder if the determined level of the CFB-CAP is elevated as compared to the control level; or (b) the patient has regression of the disease or disorder if the determined level of the CFB-CAP is descreased as compared to the control level. In some embodiments, the disease or disorder is an autoimmune disease or inflammation. In some embodiments, the disease or disorder is systemic lupus erythematosus.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1A and 1B are a set of diagrams showing a method for CFB-CAP detection by capillary flow using a “dipstick” assay.

FIGS. 2A and 2B are a set of diagrams showing a method for CFB-CAP detection by capillary flow using a lateral flow assay (LFA). FIG. 2A is a schematic representation of an LFA assay in a multistrip format for detecting CFB-CAPs, such as BC4d, TC4d, EC4d, PC4d, RC4d, and GC4d. FIG. 2B a schematic representation of an LFA assay in a multiplex format.

FIGS. 3A, 3B, and 3C are a set of diagrams showing CFB-CAP detection using an LFA assay. FIG. 3A shows detection of purified human C4d using an LFA assay. FIG. 3B shows detection of C4d in freeze-thawed buffy coat lysates. FIG. 3C shows detection of C4d in freeze-thawed red blood cell (RBC) lysates.

FIG. 4 is a diagram showing measurements of the results of a capillary flow assay using Image J. The results of the capillary assays were visualized and semi-quantitatively analyzed based on test line intensities.

FIGS. 5A, 5B, 5C, 5D, and 5E are a set of diagrams showing quantitation of the results of an LFA assay for purified C4d and correlation of the results of the LFA assay with those of other assays (e.g., flow cytometry) for different complement activation products. FIG. 5A shows quantitation of the results of an LFA assay for purified C4d to determine the correlation between C4d levels and line intensities. FIG. 5B shows correlation of EC4d levels determined by flow cytometry with those determined by the capillary flow assay. FIG. 5C shows the correlation of TC4d levels determined by flow cytometry with those determined by the capillary flow assay.

FIG. 5D shows the correlation of BC4d levels determined by flow cytometry with those determined by the capillary flow assay. FIG. 5E shows the correlation of EC4d levels in freeze-thawed samples of RBCs determined by flow cytometry with those determined by capillary flow assay. FIG. 5F shows the correlation of EC4d, BC4d, and TC4d as measured by flow cytometry (cells), ELISA (cell lysates), and LFA (cell lysates).

FIGS. 6A, 6B, and 6C are a set of diagrams showing example applications of the disclosed methods for multiplex detection. FIG. 6A shows detection of anti-lymphocyte autoantibodies (ALA) in patient plasma by a lateral flow assay (LFA). FIG. 6B shows detection of CFB-CAPs and anti-lymphocyte autoantibodies (ALA) by a duplexed LFA. FIG. 6C shows detection of erythrocyte-bound C4d (E-C4d) and plasma C4/C4b/C4d by an LFA.

### DETAILED DESCRIPTION

This disclosure provides a novel method for detecting one or more complement activation products, such as those attached to a cell fragment (referred to as cell fragment-bound complement activation products (“CFB-CAPs”), using a capillary flow system. The method eliminates the need for fresh, live cellular samples and detection by flow cytometric methods. The method, as disclosed, has a wide variety of applications, including diagnosing or monitoring lupus or pre-lupus and other diseases or disorders (e.g., autoimmune or inflammatory diseases or disorders).

This disclosure incorporates the disclosures of the following patents or patent publications by reference in their entirety: US20190302112, U.S. Pat. No. 9,863,946, US20170030905, U.S. Pat. No. 9,709,564, US20150339449, US20120122241, US20110275060, US20100233752, US20080131914, U.S. Pat. Nos. 7,361,517, 8,080,382, 7,390,631, WO2014093268, and WO2007033369.

In one aspect, this disclosure provides a method of determining a level of a complement activation product (such as CFB-CAP) in a fluid path (such as a test strip). In some embodiments, the method may comprise: (i) contacting a sample comprising one or more analytes with a detection agent, wherein the one or more analytes comprise a cell fragment and a CFB-CAP (such as C4d), and wherein the detection agent comprises a detection antibody (such as an anti-C4d antibody) that specifically binds to the CFB-CAP and facilitates detection of the CFB-CAP in at least one of the one or more analytes; (ii) immobilizing the at least one of the one or more analytes in a fluid path; and (iii) determining a level of the CFB-CAP in the at least one of the one or more analytes at one or more locations in the fluid path by determining a level of binding of the detection antibody to the CFB-CAP. In some embodiments, the sample comprises a blood sample.

The term “cell fragment-bound complement activation product,” or “CFB-CAP,” as used herein, refers to a complement activation product that is attached to a cell fragment, such as a cell fragment of a blood cell (including, but not limited to, an erythrocyte, reticulocyte, T lymphocyte, B lymphocyte, monocyte, granulocyte, eosinophil, basophil or platelet). As used in this disclosure, a complement activation product is derived from a “complement pathway component” that includes proteins from the classical, alternative, and lectin complement pathways, e.g., C1, C4, C2, C3 and fragments thereof, e.g., C4a, C4b, C2a, C2b, C4b, C2a, C3a, C3b, C4c, C4d, iC4b, C3d, C3i, C3dg. Also included are C5, C5b, C6, C7, C8, C9, C1inh, MASP1, MASP2, MBL, MAC, CR1, DAF, MCP, C4 binding protein (C4BP), Factor H, Factor B, C3bB, Factor D, Bb, Ba, C3bBb, properdin, C3bBb, CD59, C3aR, C5aR, C1qR, CR2, CR3, and CR4, as well as other complement pathway components, receptors and ligands not listed specifically herein. A CFB-CAP may be attached to a cell fragment contained in cell lysates of a cell, such as a blood cell (including, but not limited to, an erythrocyte, reticulocyte, T lymphocyte, B lymphocyte, monocyte, granulocyte, eosinophil, basophil or platelet). In some embodiments, the CFB-CAP is attached to at least a fragment (such as a cell fragment) of erythrocytes, lymphocytes, reticulocytes, platelets, granulocytes, monocytes, eosinophils, or basophils.

In some embodiments, the CB-CAP comprises a complement activation product selected from BC4d, TC4d, EC4d, PC4d, RC4d, GC4d, MC4d, and combinations thereof.

In some embodiments, the sample comprises at least one entity selected from erythrocytes, lymphocytes, reticulocytes, platelets, granulocytes, monocytes, eosinophils, and basophils, and the method comprises lysing the at least one entity in the sample before contacting the sample with the detection agent.

In some embodiments, the sample subject to CFB-CAP detection may include a cell lysate. A cell lysate may be prepared from cells such as erythrocytes, lymphocytes, reticulocytes, platelets, granulocytes, monocytes, eosinophils, or basophils. In some embodiments, a cell lysate may be prepared by lysing cells such as erythrocytes, lymphocytes, reticulocytes, platelets, granulocytes, monocytes, eosinophils, or basophils, for example, by contacting the cells with a lysis reagent (such as a lysis buffer).

In some embodiments, the sample, after being obtained from the patient, has been left at a temperature equal to or above 4 degrees Celsius for a least a period of time before being contacted with the detection agent. In some embodiments, at least a portion of the entity has been spontaneously lysed. In some embodiments, the method comprises holding the sample at a temperature equal to or above 4 degrees Celsius (such as 16, 20, 25, 30, or 36 degrees Celsius) for a period of time before contacting the sample with the detection agent, whereby at least a portion of the entity is spontaneously lysed. In some embodiments, the period time is about or greater than 60 minutes (such as 60, 70, 80, 90, 100, 110, 120, 140, 160, 180, 200, 220, or 240 minutes; 5, 12, 24, 36, or 48 hours; 3, 4, 5, 6, 7, 8, 9, 10, 11, 12, 13, or 14 days; 3, 4, 5, 6, 7, or 8 weeks).

In some embodiments, the method further comprises fixing the one or more analytes in the sample before contacting the sample with the detection agent. The term “fixing” or “fixation” as used herein is the process of preserving biological material (such as cells or cell fragments) from decay and/or degradation. Fixation may be accomplished using any convenient protocol. Fixation can include contacting the cellular sample with a fixation reagent (i.e., a reagent that contains at least one fixative). Cellular samples can be contacted by a fixation reagent for a wide range of times, which can depend on the temperature, the nature of the sample, and on the fixative(s). For example, a cellular sample can be contacted by a fixation reagent for 24 or less hours, 18 or less hours, 12 or less hours, 8 or less hours, 6 or less hours, 4 or less hours, 2 or less hours, 60 or less minutes, 45 or less minutes, 30 or less minutes, 25 or less minutes, 20 or less minutes, 15 or less minutes, 10 or less minutes, 5 or less minutes, or 2 or less minutes. Any convenient fixation reagent can be used. Common fixation reagents include cross-linking fixatives, precipitating fixatives, oxidizing fixatives, mercurials, and the like. Crosslinking fixatives chemically join two or more molecules by a covalent bond and a wide range of cross-linking reagents can be used. Examples of suitable cross-linking fixatives include but are not limited to aldehydes (e.g., formaldehyde, also commonly referred to as “paraformaldehyde” and “formalin”; glutaraldehyde; etc.), imidoesters, NHS (N-Hydroxysuccinimide) esters, and the like.

In some embodiments, the CFB-CAP comprises a cell fragment-bound C4d. In some embodiments, the detection antibody comprises an anti-C4d antibody. In some embodiments, the cell fragment-bound C4d is a complement activation product selected from BC4d, TC4d, EC4d, PC4d, RC4d, GC4d, MC4d, and combinations thereof.

In some embodiments, the detection agent further comprises enzyme substrates or chemiluminescent substrates. In some embodiments, detecting binding of the detection antibody to the CFB-CAP comprises detecting a chemiluminescent signal.

In some embodiments, the detection antibody comprises a label. In some embodiments, the label comprises a nanoparticle label, a fluorescent label, a chemiluminescent label, a radiolabel, or an enzyme.

In some embodiments, the at least one of the one or more analytes is immobilized in the fluid path by a capture antibody (such as an anti-C4d antibody). In some embodiments, the capture antibody binds to a different epitope on the CFB-CAP from one to which the detection antibody binds or binds to the detection antibody. In some embodiments, the capture antibody is an anti-C4d antibody.

In some embodiments, the capture antibody comprises a tag or a moiety that immobilizes the capture antibody in the fluid path.

In some embodiments, detection of the CFB-CAP in a fluid path can be carried out in a different way. In one example, a sample comprising one or more analytes comprising a CFB-CAP (such as C4d) is mixed with a capture antibody and a detection agent comprising a detection antibody (such as anti-C4d antibody) to obtain a reaction mixture. The capture antibody and the detection antibody may bind to distinct epitopes on the CFB-CAP, thus allowing the capture antibody and the detection antibody to simultaneously bind to the CFB-CAP. The capture antibody may include a tag or a reactive moiety that facilitates immobilization of the capture antibody to the fluid path. The detection antibody may include a label that facilitates detection of binding of the detection antibody to one or more analytes. The reaction mixture can be loaded to the fluid path to allow the complexes formed of the capture antibody, the one or more analytes, and the detection antibody to be immobilized in the fluid path for subsequent detection.

In another example, the capture antibody may be immobilized in the fluid path prior to loading the sample comprising one or more analytes or the detection agent. The sample and the detection agent can then be loaded together or sequentially to the fluid path. In yet another example, the capture antibody and the detection antibody are loaded to the fluid path before the sample.

In some embodiments, immobilizing at least one of the one or more analytes in a fluid path is performed before contacting the sample with the detection agent.

In some embodiments, the fluid path comprises a test strip that comprises a substrate formed of a porous material or a wicking material. In some embodiments, the sample comprising one or more analytes or the detection agent can be conveyed along the fluid path by capillary action. The term “capillary action” or “capillary force,” as used herein, refers to the force that results from adhesive forces and surface tension acting on a fluid in a small passage or vessel, such as a tube, which serves to move a fluid through the vessel (which may be a substrate or a capillary tube within a substrate). When the adhesive force generated by intermolecular attraction between fluid molecules and the walls of a vessel in which the fluid is contained is stronger than the cohesive forces within the fluid resulting from intermolecular attraction between the fluid molecules, an upward force on the fluid at the edges of the vessel results. This force pulls the fluid at the vessel edges upward, resulting in a meniscus. At the same time, surface tension generated by the enhanced cohesive forces between fluid molecules at the surface of the fluid acts to hold the surface intact, resulting in the upward movement of the entire fluid surface and not only the edges of the fluid surface. This combination of forces is referred to as capillary force or action. The term “wicking” or “wicking forces,” as used herein, refers to the movement of fluid through a porous medium as a result of capillary forces occurring in the pores of the medium. Typically, a porous medium has some degree of capillarity to the extent that fluid moves through the medium due to capillary forces created by, for example, small diameter pores or the close proximity of fibers.

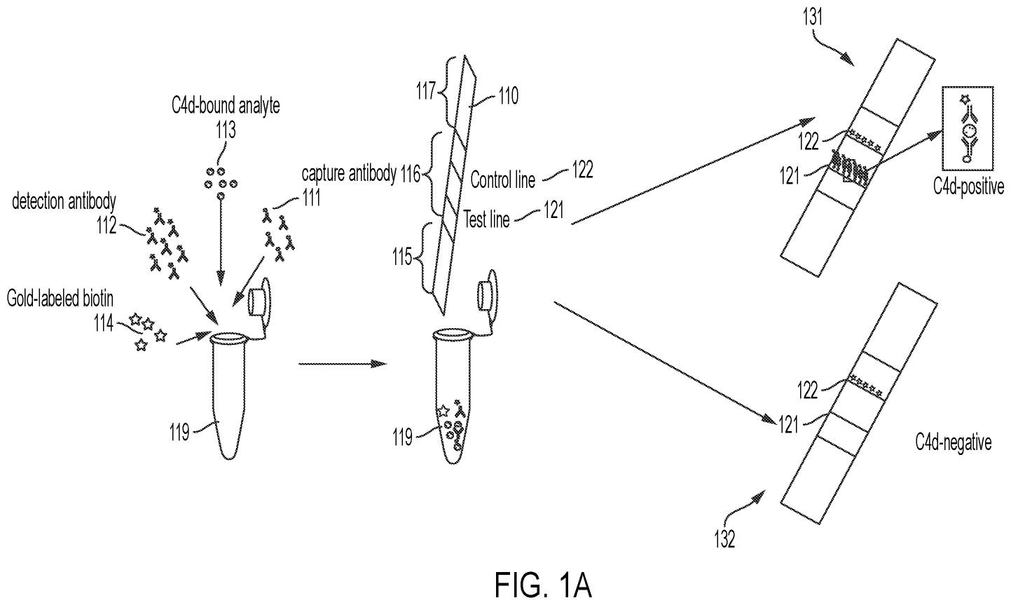

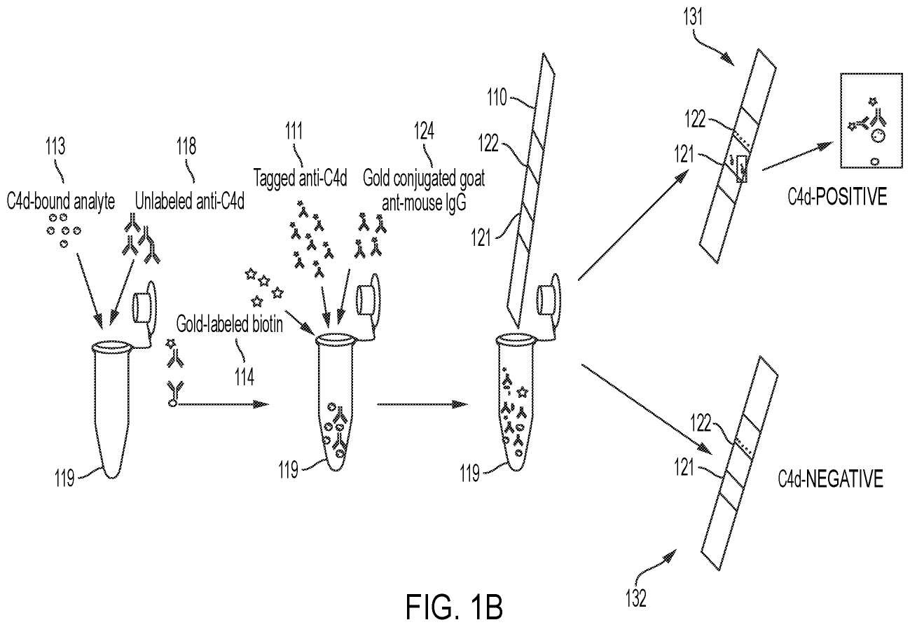

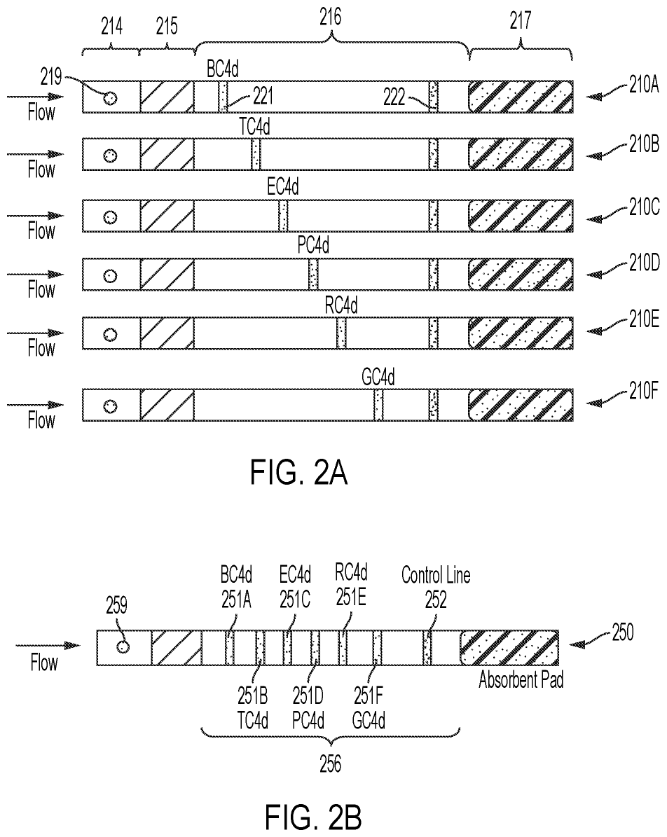

The methods described above are illustrated by way of examples in FIGS. 1A, 1B, 2A, and 2B. FIG. 1 shows a schematic representation of a method for CFB-CAP detection by a “dipstick” assay using a test strip 110, a tagged capture antibody 111 (in this case, a first C4d antibody), and a labeled detection antibody 112 (which in this example is a second C4d antibody). The capture antibody 111 and the detection antibody 112 are both anti-CFB-CAP antibodies and bind to distinct epitopes on a CFB-CAP, thus allowing simultaneous binding of the capture antibody 111 and the detection antibody 112 to the CFB-CAP 113 (in this case, a C4d bound analytic). FIG. 1B shows a schematic representation of a method for CFB-CAP detection by a “dipstick” assay using a tagged capture antibody 111 (e.g., an anti-CFB-CAP antibody, such as an anti-C4d antibody), a labeled detection antibody 112, and an unlabeled competitor antibody 118 (also an anti-CFB-CAP antibody, such as an anti-C4d antibody may be added). The labeled detection antibody 112 binds to a portion of the tagged capture antibody 111 and facilitates the detection of a CFB-CAP. Example 1 below describes FIG. 1A in more detail.

In some embodiments, the detection agent further comprises enzyme substrates or chemiluminescent substrates. In some embodiments, detecting binding of the antibody to the CFB-CAP comprises detecting a chemiluminescent signal.

In some embodiments, the antibody or the capture antibody is a bispecific antibody, a trispecific antibody, a single chain Fv (scFv), a monoclonal antibody, a chimeric antibody, a humanized antibody, a recombinant antibody, or a human antibody.

The term “antibody” (Ab) as used herein includes monoclonal antibodies, polyclonal antibodies, multi-specific antibodies (for example, bispecific antibodies and polyreactive antibodies), and antibody fragments. Thus, the term “antibody” as used in any context within this specification is meant to include, but not be limited to, any specific binding member, immunoglobulin class and/or isotype (e.g., IgG1, IgG2, IgG3, IgG4, IgM, IgA, IgD, IgE, and IgM); and biologically relevant fragment or specific binding member thereof, including but not limited to Fab, F(ab′)2, Fv, and scFv (single chain or related entity). It is understood in the art that an antibody is a glycoprotein having at least two heavy (H) chains and two light (L) chains inter-connected by disulfide bonds, or an antigen-binding portion thereof. A heavy chain is comprised of a heavy chain variable region (VH) and a heavy chain constant region (CH1, CH2, and CH3). A light chain is comprised of a light chain variable region (VL) and a light chain constant region (CL). The variable regions of both the heavy and light chains comprise framework regions (FWR) and complementarity determining regions (CDR). The four FWR regions are relatively conserved while CDR regions (CDR1, CDR2, and CDR3) represent hypervariable regions and are arranged from NH2 terminus to the COOH terminus as follows: FWR1, CDR1, FWR2, CDR2, FWR3, CDR3, and FWR4. The variable regions of the heavy and light chains contain a binding domain that interacts with an antigen while, depending on the isotype, the constant region(s) may mediate the binding of the immunoglobulin to host tissues or factors.

Also included in the definition of “antibody” as used herein are chimeric antibodies, humanized antibodies, and recombinant antibodies, human antibodies generated from a transgenic non-human animal, as well as antibodies selected from libraries using enrichment technologies available to the artisan.

The term “variable” refers to the fact that certain segments of the variable (V) domains differ extensively in sequence among antibodies. The V domain mediates antigen-binding and defines specificity of a particular antibody for its particular antigen. However, the variability is not evenly distributed across the amino acid span of the variable regions. Instead, the V regions consist of relatively invariant stretches called framework regions (FRs) of 15-30 amino acids separated by shorter regions of extreme variability called “hypervariable regions” that may be 9-12 amino acids long. The variable regions of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see, for example, Kabat et al., Sequences of Proteins of Immunological Interest, 5th Ed. Public Health Service, National Institutes of Health, Bethesda, Md. (1991)). The term “hypervariable region,” as used herein, refers to the amino acid residues of an antibody that are responsible for antigen binding. The hypervariable region generally comprises amino acid residues from a “complementarity determining region” (“CDR”).

The term “monoclonal antibody” as used herein refers to an antibody obtained from a population of substantially homogeneous antibodies, i.e., the individual antibodies comprising the population are identical except for possible naturally occurring mutations that may be present in minor amounts. The term “polyclonal antibody” refers to preparations that include different antibodies directed against different determinants (“epitopes”).

The monoclonal antibodies herein include “chimeric” antibodies in which a portion of the heavy and/or light chain is identical with, or homologous to, corresponding sequences in antibodies derived from a particular species or belonging to a particular antibody class or subclass, while the remainder of the chain(s) is identical with, or homologous to, corresponding sequences in antibodies derived from another species or belonging to another antibody class or subclass, as well as fragments of such antibodies, so long as they exhibit the desired biological activity (see, for example, U.S. Pat. No. 4,816,567; and Morrison et al., Proc. Natl. Acad. Sci. USA, 81:6851-6855 (1984)). The variable region antigen-binding sequences can be derived from human or non-human antibodies. For example, chimeric antibodies may include antibodies having one or more non-human antigen-binding sequences (for example, CDRs) and containing one or more sequences derived from a human antibody, for example, an FR or C region sequence. In addition, chimeric antibodies included herein are those comprising a human or non-human variable region antigen-binding sequence of one antibody class or subclass and another sequence, for example, FR or C region sequence, derived from another antibody class or subclass.

A “humanized antibody” generally is considered to be a human antibody that has one or more amino acid residues introduced into it from a source that is non-human. These non-human amino acid residues often are referred to as “import” residues, which typically are taken from an “import” variable region. Humanization may be performed following the method of Winter and co-workers (see, for example, Jones et al., Nature, 321:522-525 (1986); Reichmann et al., Nature, 332:323-327 (1988); Verhoeven et al., Science, 239:1534-1536 (1988)), by substituting import hypervariable region sequences for the corresponding sequences of a human antibody. Accordingly, such “humanized” antibodies are chimeric antibodies (see, for example, U.S. Pat. No. 4,816,567), where substantially less than an intact human variable region has been substituted by the corresponding sequence from a non-human species.

An “antibody fragment” comprises a portion of an intact antibody, such as the antigen-binding or variable region of the intact antibody. Examples of antibody fragments include, but are not limited to, Fab, Fab′, F(ab′)2, and Fv fragments; diabodies; linear antibodies (see, for example, U.S. Pat. No. 5,641,870; Zapata et al., Protein Eng. 8(10): 1057-1062 [1995]); single-chain antibody molecules; and multi-specific antibodies formed from antibody fragments.

“Fv” is the minimum antibody fragment that contains a complete antigen-recognition and antigen-binding site. This fragment contains a dimer of one heavy- and one light-chain variable region domain in tight, non-covalent association. From the folding of these two domains emanate six hypervariable regions (three loops each from the H and L chain) that contribute the amino acid residues for antigen-binding and confer antigen-binding specificity to the antibody. However, even a single variable region (or half of an Fv comprising only three CDRs specific for an antigen) has the ability to recognize and bind antigen, although at a lower affinity than the entire binding site. “Single-chain Fv” (“sFv” or “scFv”) are antibody fragments that comprise the VH and VL antibody domains connected into a single polypeptide chain. The sFv polypeptide can further comprise a polypeptide linker between the VH and VL domains that enables the sFv to form the desired structure for antigen binding. For a review of sFv, see, for example, Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments with short linkers (about 5-10 residues) between the VH and VL domains such that inter-chain but not the intra-chain pairing of the V domains is achieved, resulting in a bivalent fragment, i.e., fragment having two antigen-binding sites. Bispecific diabodies are heterodimers of two “crossover” sFv fragments in which the VH and VL domains of the two antibodies are present on different polypeptide chains. Diabodies are described more fully in, for example, European Patent Number EP 404,097; WIPO International Patent Application Publication Number WO 93/11161; and Hollinger et al., Proc. Natl. Acad. Sci. USA, 90:6444-6448 (1993).

Domain antibodies (dAbs), which can be produced in fully human form, are the smallest known antigen-binding fragments of antibodies, ranging from about 11 kDa to about 15 kDa. DAbs are the robust variable regions of the heavy and light chains of immunoglobulins (VH and VL, respectively). They are highly expressed in microbial cell culture, show favorable biophysical properties including, for example, but not limited to, solubility and temperature stability, and are well suited to selection and affinity maturation by in vitro selection systems such as, for example, phage display. DAbs are bioactive as monomers and, owing to their small size and inherent stability, can be formatted into larger molecules to create drugs with prolonged serum half-lives or other pharmacological activities. Examples of this technology have been described in, for example, WIPO International Patent Application Publication Number WO9425591 for antibodies derived from Camelidae heavy chain Ig, as well in U.S. Patent Application Publication Number US20030130496, describing the isolation of single domain fully human antibodies from phage libraries.

Fv and sFv are the only species with intact combining sites that are devoid of constant regions. Thus, they are suitable for reduced nonspecific binding during in vivo use. sFv fusion proteins can be constructed to yield fusion of an effector protein at either the amino or the carboxy terminus of an sFv. See, for example, Antibody Engineering, ed. Borrebaeck, supra. The antibody fragment also can be a “linear antibody,” for example, as described in U.S. Pat. No. 5,641,870 for example. Such linear antibody fragments can be monospecific or bispecific.

In some embodiments, antibodies used/described in this disclosure are bispecific or multi-specific. Bispecific antibodies are antibodies that have binding specificities for at least two different epitopes. Exemplary bispecific antibodies can bind to two different epitopes of a single antigen. Other such antibodies can combine a first antigen-binding site with a binding site for a second antigen. Bispecific antibodies also can be used to localize cytotoxic agents to infected cells. Bispecific antibodies can be prepared as full-length antibodies or antibody fragments (for example, F(ab′)2 bispecific antibodies).

In some embodiments, the bispecific antibody comprises a first antigen-binding arm binding to C4d and a second antigen-binding arm binding to any one of CD3, CD4, CD5, CD8, CD45, CD19, CD20, CD21, CD22, CD23, CD25, CD40, CD42b, CD69, CD70, CD79, CD80, CD85, CD86, CD137, CD138, CD252, and CD268. In some embodiments, the chimeric antibody comprises a human Fc domain and a murine variable region.

Methods for making bispecific antibodies are known in the art. Traditional production of full-length bispecific antibodies is based on the co-expression of two immunoglobulin heavy chain-light chain pairs, where the two chains have different specificities (see, for example, Millstein et al., Nature, 305:537-539 (1983)). Similar procedures are disclosed in, for example, WO 93/08829, Traunecker et al., EMBO J., 10:3655-3659 (1991) and see also Mouquet et al., Enhanced HIV-1 neutralization by antibody heteroligation” Proc Natl Acad Sci USA. 2012 Jan. 17; 109(3):875-80.

Alternatively, antibody variable regions with the desired binding specificities (antibody-antigen combining sites) are fused to immunoglobulin constant domain sequences. The fusion is with an Ig heavy chain constant domain, comprising at least part of the hinge, CH2, and CH3 regions. According to some embodiments, the first heavy-chain constant region (CH1) containing the site necessary for light chain bonding, is present in at least one of the fusions. DNAs encoding the immunoglobulin heavy chain fusions and, if desired, the immunoglobulin light chain, are inserted into separate expression vectors and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

Techniques for generating bispecific antibodies from antibody fragments also have been described in the literature. For example, bispecific antibodies can be prepared using chemical linkage. For example, Brennan et al., Science, 229: 81 (1985) describe a procedure wherein intact antibodies are proteolytically cleaved to generate F(ab′)2 fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives then is reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Other modifications of the antibody are contemplated herein. For example, the antibody can be linked to one of a variety of nonproteinaceous polymers, for example, polyethylene glycol, polypropylene glycol, polyoxyalkylenes, or copolymers of polyethylene glycol and polypropylene glycol. The antibody also can be entrapped in microcapsules prepared, for example, by coacervation techniques or by interfacial polymerization (for example, hydroxymethyl cellulose or gelatin-microcapsules and poly-(methylmethacrylate) microcapsules, respectively), in colloidal drug delivery systems (for example, liposomes, albumin microspheres, microemulsions, nanoparticles, and nanocapsules), or in macroemulsions. Such techniques are disclosed in, for example, Remington's Pharmaceutical Sciences, 16th edition, Oslo, A., Ed., (1980).

Typically, the antibodies can be produced recombinantly, using vectors and methods available in the art. Human antibodies also can be generated by in vitro activated B cells (see, for example, U.S. Pat. Nos. 5,567,610 and 5,229,275). General methods in molecular genetics and genetic engineering useful in the present disclosure are described in the current editions of Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutscher, ed., (1990) Academic Press, Inc.); PCR Protocols: A Guide to Methods and Applications (Innis et al. 1990. Academic Press, San Diego, CA), Culture of Animal Cells: A Manual of Basic Technique, 2nd Ed. (R. I. Freshney. 1987. Liss, Inc. New York, NY), and Gene Transfer and Expression Protocols, pp. 109-128, ed. E. J. Murray, The Humana Press Inc., Clifton, N.J.). Reagents, cloning vectors, and kits for genetic manipulation are available from commercial vendors, such as BioRad, Stratagene, Invitrogen, ClonTech, and Sigma-Aldrich Co.

Human antibodies also can be produced in transgenic animals (for example, mice) that are capable of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice results in the production of human antibodies upon antigen challenge. See, for example, Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806, 5,569,825, 5,591,669 (all of GenPharm); U.S. Pat. No. 5,545,807; and WIPO International Patent Application Publication No. WO 97/17852. Such animals can be genetically engineered to produce human antibodies comprising a polypeptide of the described invention.

Various techniques have been developed for the production of antibody fragments. Traditionally, these fragments were derived via proteolytic digestion of intact antibodies (see, for example, Morimoto et al., Journal of Biochemical and Biophysical Methods 24:107-117 (1992); and Brennan et al., Science, 229:81 (1985)). However, these fragments can now be produced directly by recombinant host cells. Fab, Fv, and ScFv antibody fragments can all be expressed in and secreted from E. coli, thus allowing the facile production of large amounts of these fragments. Fab′-SH fragments can be directly recovered from E. coli and chemically coupled to form F(ab′)2 fragments (see, for example, Carter et al., Bio/Technology 10:163-167 (1992)). According to another approach, F(ab′)2 fragments can be isolated directly from recombinant host cell culture. Fab and F(ab′)2 fragments with increased in vivo half-life comprising a salvage receptor binding epitope residues are described in U.S. Pat. No. 5,869,046. Other techniques for the production of antibody fragments will be apparent to the skilled practitioner.

Other techniques that are known in the art for the selection of antibody fragments from libraries using enrichment technologies, including but not limited to phage display, ribosome display (Hanes and Pluckthun, 1997, Proc. Nat. Acad. Sci. 94: 4937-4942), bacterial display (Georgiou, et al., 1997, Nature Biotechnology 15: 29-34) and/or yeast display (Kieke, et al., 1997, Protein Engineering 10: 1303-1310) may be utilized as alternatives to previously discussed technologies to select single chain antibodies. Single-chain antibodies are selected from a library of single chain antibodies produced directly utilizing filamentous phage technology. Phage display technology is known in the art (e.g., see technology from Cambridge Antibody Technology (CAT)) as disclosed in U.S. Pat. Nos. 5,565,332; 5,733,743; 5,871,907; 5,872,215; 5,885,793; 5,962,255; 6,140,471; 6,225,447; 6,291650; 6,492,160; 6,521,404; 6,544,731; 6,555,313; 6,582,915; 6,593,081, as well as other U.S. family members, or applications which rely on priority filing GB 9206318, filed 24 May 1992; see also Vaughn, et al. 1996, Nature Biotechnology 14: 309-314). Single chain antibodies may also be designed and constructed using available recombinant DNA technology, such as a DNA amplification method (e.g., PCR), or possibly by using a respective hybridoma cDNA as a template.

As used herein, the term “specific binding” or “specifically binds,” when used to describe the binding reaction between an antibody to a protein (e.g., C4d), refers to the characteristic of the binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies raised to a component of the complement pathway or to a surface marker of platelets, polymorphic variants, alleles, orthologs, and conservatively modified variants, or splice variants, or portions thereof, can be selected to obtain only those polyclonal antibodies that are specifically immunoreactive with the component of the complement pathway or the platelet surface marker and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

In another aspect, this disclosure also provides a kit for determining a level of one or more CFB-CAPs (e.g., C4d). In some embodiments, the kit may include (i) a detection agent comprising at least one anti-CFB-CAP antibody (e.g., anti-C4d antibody); (ii) at least one test strip or at least one capillary tube; and (iii) optionally an apparatus for collecting a sample (e.g., bodily fluid). In some embodiments, the apparatus for collecting a sample may include, without limitation, a capillary tube, a pipette, a syringe, a needle, a pump, and a swab. In some embodiments, the kit may include an informational material. The informational material can be descriptive, instructional, marketing or other material that relates to the methods described herein. In some embodiments, the kit also includes an additional agent contained in the same or different container from the detection agent. For example, the kit may include a capture antibody provided in a separate container or a separate compartment from the detection agent.

In another aspect, the methods as disclosed herein can be used for multiplex detection of CFB-CAPs and one or more additional biological entities, for example, in a single strip or in two or more separate strips. Additional biological entities may include freely circulating complement activation products (FC-CAPs) such as plasma circulating complement activation products, antibodies, nucleic acids, proteins, cells or fragments thereof. Accordingly, two or more capture antibodies may be used to immobilize CFB-CAPs and one or more additional biology entities at at least one location in a fluid path.

The term “freely circulating complement activation products,” or “FC-CAPs” as used herein, refers to a complement activation product that is not attached to a cell or a cell fragment, such as a cell fragment of a blood cell (including, but not limited to, an erythrocyte, reticulocyte, T lymphocyte, B lymphocyte, monocyte, granulocyte, eosinophil, basophil or platelet). The term, “freely circulating complement activation products,” or “FC-CAPs,” as used herein, includes plasma circulating complement activation products. As used in this disclosure, a complement activation product is derived from a “complement pathway component” that includes proteins from the classical, alternative, and lectin complement pathways, e.g., C1, C4, C2, C3 and fragments thereof, e.g., C4a, C4b, C2a, C2b, C4b, C2a, C3a, C3b, C4c, C4d, iC4b, C3d, C3i, C3dg. Also included are C5, C5b, C6, C7, C8, C9, Clinh, MASP1, MASP2, MBL, MAC, CR1, DAF, MCP, C4 binding protein (C4BP), Factor H, Factor B, C3bB, Factor D, Bb, Ba, C3bBb, properdin, C3bBb, CD59, C3aR, C5aR, C1qR, CR2, CR3, and CR4, as well as other complement pathway components, receptors and ligands not listed specifically herein.

In one example, the one or more analytes may include a CFB-CAP and a FC-CAP. Accordingly, a first capture antibody that binds the CFB-CAP and a second capture antibody that binds to the FC-CAP may be used to immobilize the one or more analytes for detection of the CFB-CAP and/or the FC-CAP.

In another example, the one or more analytes may include a CFB-CAP and an anti-T cell antibody (such as anti-T cell autoantibody). Accordingly, a first capture antibody that binds the CFB-CAP and a second capture antibody that binds to the anti-T cell antibody may be used to immobilize the one or more analytes for detection of the CFB-CAP and/or the anti-T cell antibody.

The terms, “anti-T cell antibody,” “anti-lymphocyte autoantibodies (ALA),” and “anti-T cell autoantibodies” are used interchangeably herein.

In yet another example, the one or more analytes may include a CFB-CAP, a FC-CAP, and an anti-T cell antibody. Accordingly, a first capture antibody that binds to the CFB-CAP, a second capture antibody that binding to the FC-CAP, and a third capture antibody that binds to the anti-T cell antibody may be used to immobilize the one or more analytes for detection of the CFB-CAP, the FC-CAP, and/or the anti-T cell antibody.

In some embodiments, the disclosed methods can be used for simultaneous detection of an anti-T cell antibody and a CFB-CAP with a single LFA test strip. In some embodiments, the disclosed methods can be used for simultaneous detection of a FC-CAP and a CFB-CAP with a single LFA test strip. In some embodiments, the disclosed methods can be used for simultaneous detection of an anti-T cell antibody, a CFB-CAP, and a FC-CAP with a single LFA test strip.

In some embodiments, the one or more analytes further comprise an anti-T cell antibody. In some embodiments, the method comprises determining a level of at least one of the CFB-CAP and the anti-T cell antibody in the one or more analytes. In some embodiments, the method comprises determining a level of each of the CFB-CAP and the anti-T cell antibody in the one or more analytes.

In some embodiments, the CFB-CAP is attached to at least a fragment (such as a cell fragment) of erythrocytes, lymphocytes, reticulocytes, platelets, granulocytes, monocytes, eosinophils, or basophils. In some embodiments, the CFB-CAP is a complement activation product selected from BC4d, TC4d, EC4d, PC4d, RC4d, GC4d, MC4d, and combinations thereof.

In some embodiments, the capture antibody comprises a first capture antibody that binds to the CFB-CAP and a second capture antibody that binds to the anti-T cell antibody. In some embodiments, the first capture antibody and the second capture antibody are configured to immobilize the CFB-CAP and the anti-T cell antibody at at least two separate locations in the fluid path. In some embodiments, the anti-T cell antibody is an anti-T cell autoantibody.

In some embodiments, the one or more analytes further comprise a FC-CAP.

In some embodiments, the method comprises determining a level of each of the CFB-CAP and the FC-CAP in the one or more analytes. In some embodiments, the capture antibody comprises a first capture antibody that binds to the CFB-CAP and a second capture antibody that binds to the FC-CAP. In some embodiments, the first capture antibody and the second capture antibody are configured to immobilize the CFB-CAP and the FC-CAP at at least two separate locations in the fluid path.

In some embodiments, the one or more analytes further comprise an anti-T cell antibody. In some embodiments, the method comprises determining a level of at least one of the CFB-CAP, the FC-CAP, and the anti-T cell antibody in the one or more analytes. In some embodiments, the method comprises determining a level of each of the CFB-CAP, the FC-CAP, and the anti-T cell antibody in the one or more analytes. In some embodiments, the capture antibody comprises a first capture antibody that binds to the CFB-CAP, a second capture antibody that binds to the FC-CAP, and a third capture antibody that binds to the anti-T cell antibody, wherein the first capture antibody, the second capture antibody, and the third capture antibody are configured to immobilize the CFB-CAP, the FC-CAP, and the anti-T cell antibody at two or more separate locations (such as three separate locations) in the fluid path.

In some embodiments, the CFB-CAP is attached to at least a fragment (such as a cell fragment) of erythrocytes, lymphocytes, reticulocytes, platelets, granulocytes, monocytes, eosinophils, or basophils.

In some embodiments, the CFB-CAP or the FC-CAP is a complement activation product selected from BC4d, TC4d, EC4d, PC4d, RC4d, GC4d, MC4d, and combinations thereof.

In another aspect, this disclosure provides a method of identifying a patient as exhibiting lupus or pre-lupus. The method comprises: (i) obtaining a sample (such as a blood sample) for the patient; (ii) determining a level of the CFB-CAP in the sample by a method described above; (iii) comparing the determined level of the CFB-CAP with a control level and determining whether the determined level is elevated as compared to the control level; and (iv) determining that the patient has lupus or an increased risk of developing lupus if the determined level of the CFB-CAP is elevated as compared to the control level.

In some embodiments, the method further comprises: (a) determining a level of an anti-T cell antibody contained in the blood sample by the method as described herein; (b) comparing the determined level of the anti-T cell antibody with a second control level and determining whether the determined level of the anti-T cell antibody is elevated as compared to the second control level; and (c) determining that the patient has lupus or an increased risk of developing lupus if the determined level of the CFB-CAP and the determined level of the anti-T cell antibody are elevated as compared to the control level and the second control level, respectively.

As used herein, “lupus,” “systemic lupus erythematosus,” or “SLE” is a prototypic autoimmune disease resulting in multiorgan involvement. This anti-self response is characterized by autoantibodies directed against a variety of nuclear and cytoplasmic cellular components. These autoantibodies bind to their respective antigens, forming immune complexes that circulate and eventually deposit in tissues. This immune complex deposition and consequential activation of the complement system causes chronic inflammation and tissue damage. Lupus progresses in a series of flares, or periods of acute illness, followed by remissions. The symptoms of a lupus flare, which vary considerably among patients and even within the same patient, include malaise, fever, joint pain, and photosensitivity (development of rashes after brief sun exposure). Other symptoms of lupus include hair loss, ulcers of mucous membranes, inflammation of the lining of the heart and lungs, which leads to chest pain, and synovitis, a painful inflammation of synovial membranes. Red blood cells, platelets, and white blood cells can be targeted in lupus, resulting in anemia, bleeding, and thrombotic problems. More seriously, immune complex deposition and chronic inflammation in the glomerulus can lead to kidney involvement and occasionally failure requiring dialysis or kidney transplantation. Since the blood vessel is a major target of the autoimmune response in lupus, premature strokes and heart disease are not uncommon. Over time, however, these flares can lead to irreversible organ damage. The term “lupus” may also apply to other types of lupus, such as discoid lupus erythematosus or drug-induced lupus.

As used in this document, the term “pre-lupus” refers to a classification or pre-existing condition that may serve as a preliminary indicator that a patient is at increased risk of developing lupus. A patient diagnosed with pre-lupus will have certain characteristics that would correspond to definite lupus, but has not yet developed or been diagnosed with definite lupus. The pre-lupus condition might be considered an equivalent of a precancerous or premalignant condition, which is a state associated with a significantly increased risk of developing cancer or malignancy that should be treated accordingly. Examples of precancerous or premalignant states include colon polyps, associated with an increased risk of developing colon cancer, Barrett's esophagus, associated with an increased risk of developing esophageal cancer, cervical dysplasia, associated with an increased risk of developing cervical cancer, actinic keratosis, associated with an increased risk of developing skin cancer, and premalignant lesions of the breast, associated with an increased risk of developing breast cancer. In the majority of precancerous states, treatment of the lesion reduces or eliminates the risk of developing cancer. As such, early detection is essential. The pre-lupus condition can be viewed in a similar context. Patients with pre-lupus are at increased risk of developing definite lupus, however, they may not. Early detection and appropriate treatment are essential to reducing the risk of disease progression.

The terms “patient,” “individual,” and “subject” are used interchangeably and generally refer to any living organism to which the disclosed methodology is utilized to obtain a bodily fluid sample in order to perform a diagnostic or monitoring method described herein. A patient can be an animal, such as a human. A patient may also be a domesticated animal or a farm animal. A “patient” or “individual” may also be referred to as a subject.

As used herein, a “control” level of any CFB-CAP refers, in some embodiments, to a level of that CFB-CAP obtained from a sample obtained from one or more individuals who do not suffer from the autoimmune, inflammatory or other disease or disorder that is of interest in the investigation. The level may be measured on an individual-by-individual basis or on an aggregate basis such as an average. A “control” level can also be determined by analysis of a population of individuals who have the disease or disorder but are not experiencing an acute phase of the disease or disorder. A “control” cell or sample may be used to obtain such a “control” level. A “control” cell or sample may be obtained from one or more individuals who do not suffer from the autoimmune, inflammatory or other disease or disorder that is of interest in the investigation. A “control” cell or sample can also be obtained from a population of individuals who have the disease or disorder but are not experiencing an acute phase of the disease or disorder. In some embodiments, a “control” level of a respective CFB-CAP, cell or sample is from the same individual for whom a diagnosis is sought or whose condition is being monitored, but is obtained at a different time. In certain embodiments, a “control” level, sample or cell can refer to a level, sample or cell obtained from the same patient at an earlier time, e.g., weeks, months, or years earlier.

As used herein, “the determined level is elevated as compared to the control level” refers to a positive change in value from the control level.

In yet another aspect, this disclosure provides a method of identifying a disease or disorder in an individual. The method comprises: (a) obtaining a bodily fluid sample from the patient; (b) determining a level of the CFB-CAP contained in the bodily fluid sample by the method described above; (c) comparing the determined level of the CFB-CAP with a control level and determining whether the determined level is elevated as compared to the control level; and (d) determining that the patient has the disease or disorder if the determined level of the CFB-CAP is elevated as compared to the control level. In some embodiments, the disease or disorder is an autoimmune disease or inflammation. In some embodiments, the disease or disorder is systemic lupus erythematosus.

In some embodiments, the method further comprises: (a) determining a level of an anti-T cell antibody contained in the bodily fluid sample by the method as described herein; (b) comparing the determined level of the anti-T cell antibody with a second control level and determining whether the determined level of the anti-T cell antibody is elevated as compared to the second control level; and (c) determining that the patient has the disease or disorder if the determined level of the CFB-CAP and the determined level of the anti-T cell antibody are elevated as compared to the control level and the second control level, respectively.

In yet another aspect, this disclosure provides a method of monitoring progression of a disease or disorder in an individual. The method comprises: (i) obtaining a bodily fluid sample from the patient; (ii) determining a level of the CFB-CAP contained in the bodily fluid sample by the method as described herein; (iii) comparing the determined level of the CFB-CAP with a control level and determining whether the determined level is elevated or decreased as compared to the control level; and (iv) determining that (a) the patient has progression of the disease or disorder if the determined level of the CFB-CAP is elevated as compared to the control level; or (b) the patient has regression of the disease or disorder if the determined level of the CFB-CAP is decreased as compared to the control level. In some embodiments, the disease or disorder is an autoimmune disease or inflammation. In some embodiments, the disease or disorder is systemic lupus erythematosus.

As used herein, a “sample” or “bodily fluid sample” or “fluid sample” or “individual sample” or “subject sample” or “patient sample” or the like in the context of obtaining a sample from a patient, subject or individual refers to a sample which may be blood plasma, blood serum, whole blood, CSF, urine, saliva, tears, semen, colostrum or any recoverable bodily fluid as obtained from the individual for C-TM testing in one or more of the various assays disclosed herein.

As used herein, an “autoimmune or inflammatory disease or condition” refers to (i) any autoimmune disease or immune disease or condition that causes damage of organs and increased inflammation in an individual, and/or (ii) an inflammatory disease or condition being any infectious disease or condition that causes increased inflammation in an individual. “Autoimmune disease” and “immune disease” are used interchangeably. In some instances, the terms noted in this paragraph are also used interchangeably to describe a certain disease state. In some embodiments, the inflammatory disease or condition is a “chronic inflammatory disease or condition.” A chronic inflammatory disease or condition is an inflammatory condition that does not resolve after a period of weeks, months or longer. Chronic inflammatory conditions can follow an acute inflammatory condition or for some diseases or conditions can occur in the absence of an acute inflammatory disease or condition. An autoimmune or inflammatory disease or condition includes but is not limited to the following: systemic lupus erythematosus (lupus or SLE), Sjogren's syndrome, rheumatoid arthritis, vasculitis (and its specific forms such as Wegener's granulomatosis), scleroderma, myositis, serum sickness, transplant rejection, sickle cell anemia, gout, complications of pregnancy such as pre-eclampsia, multiple sclerosis, cardiovascular disease, infectious disease such as hepatitis C virus infection, etc.

Autoimmune diseases can be broadly divided into systemic and organ-specific or localized autoimmune disorders, depending on the principal clinic-pathologic features of each disease. Each of these diseases or conditions can also be described as chronic inflammatory diseases or conditions. Systemic autoimmune diseases include but are not limited to SLE, Sjogren's syndrome, scleroderma, rheumatoid arthritis, and dermatomyositis. These conditions tend to be associated with autoantibodies to antigens which are not tissue-specific. Thus although polymyositis is more or less tissue-specific in presentation, it may be included in this group because the autoantigens are often ubiquitous t-RNA synthetases. Local syndromes which affect a specific organ or tissue include but are not limited to: diabetes mellitus type 1, Hashimoto's thyroiditis, Addison's disease (endocrinologic); Celiac disease, Crohn's disease, pernicious anemia (gastrointestinal); pemphigus vulgaris, vitiligo (dermatologic); autoimmune hemolytic anemia, idiopathic thrombocytopenic purpura (haematologic) and myasthenia gravis (neurologic). The above-identified disease states are provided as a general description of numerous immune or inflammatory disease states known in the art, but are in no way intended to limit the scope of this disclosure.

As used herein, an “inflammatory disease or condition” refers to any immune disease or condition that causes increased inflammation in an individual. An inflammatory disease or condition also refers to any infectious disease or condition that causes increased inflammation in an individual. In some embodiments, the inflammatory disease or condition is a “chronic inflammatory disease or condition.” A chronic inflammatory disease or condition is an inflammatory condition that does not resolve after a period of weeks, months or longer. Chronic inflammatory conditions can follow an acute inflammatory condition, or for some diseases or conditions can occur in the absence of an acute inflammatory disease or condition. An inflammatory disease or condition includes the following: SLE, rheumatoid arthritis, vasculitis (and its specific forms such as Wegener's granulomatosis), scleroderma, myositis, serum sickness, transplant rejection, sickle cell anemia, gout, complications of pregnancy such as pre-eclampsia, multiple sclerosis, cardiovascular disease, infectious disease such as hepatitis C virus infection, etc. Each of these diseases or conditions can also be described as chronic inflammatory diseases or conditions.

The present disclosure also relates to a kit for diagnosing or monitoring lupus or pre-lupus and other diseases or disorders (e.g., autoimmune or inflammatory diseases or disorders). In some embodiments, the kit may include an apparatus and/or a reagent for determining a level of one or more CFB-CAPs (e.g., C4d). In some embodiments, the kit may include (i) a detection agent comprising at least one anti-CFB-CAP antibody (e.g., anti-C4d antibody); (ii) at least one test strip with a substrate formed of a wicking material; and (iii) optionally an apparatus for collecting a sample (e.g., bodily fluid). In some embodiments, the apparatus for collecting a sample may include, without limitation, a capillary tube, a pipette, a syringe, a needle, a pump, and a swab. In some embodiments, the kit may include an informational material. The informational material can be descriptive, instructional, marketing or other material that relates to the methods described herein. In some embodiments, the kit also includes an additional agent contained in the same or different container from the detection agent. For example, the kit may include a capture antibody provided in a separate container or a separate compartment from the detection agent.

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

“Diagnostic,” as used herein, characterizes something that identifies the presence or nature of a pathologic condition, such as SLE. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is one minus the false positive rate, where the “false positive” rate is defined as the proportion of those without the disease who test positive. While a particular diagnostic method may not provide a definitive diagnosis of a condition, it suffices if the method provides a positive indication that aids in diagnosis. The term “diagnostic” or “diagnosing” or “diagnosis” may be used interchangeably with “identify” or “identifying” or “identification.”

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

The terms “increased,” “increase” or “enhance” or “activate” are all used herein to generally mean an increase by a statically significant amount; for the avoidance of any doubt, the terms “increased,” “increase,” or “enhance” or “activate” means an increase of at least 10% as compared to a reference level, for example, an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. When used in this document, the term “exemplary” is intended to mean “by way of example” and is not intended to indicate that a particular exemplary item is preferred or required.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

To demonstrate the feasibility of detecting CFB-CAPs based on capillary flow, CFB-CAP detection by a “dipstick” assay was performed using the standard reagents and test strips that are commercially available (FIG. 1).

25 μl of whole blood was centrifuged to separate plasma from cells. Cells were washed with phosphate-buffered saline (PBS) and incubated with a hypotonic NH4CL solution to lyse RBC. Unlysed cells (mainly white blood cells) and lysed RBCs (so-called “RBC ghosts,” RBC shells devoid of hemoglobin) were collected by centrifugation, washed, and treated with a lysis buffer to generate cell lysates containing cell fragments (CF). The cell lysate (depicted as C4d-bound analyte 113 in FIGS. 1A and 1B) was incubated in a vessel 119 with a capture antibody 111 and a detection antibody 112 for C4d. The Quidel anti-C4d labeled with the tag provided in the kit was used as the capture antibody 111 and 9A10E4 anti-C4d conjugated with colloid gold (provided in the kit) was used as the detection antibody 112 to mark C4d-bearing molecules in the cell lysate. Colloid gold-labeled biotin 114 (provided in the kit) was added to the cell lysate as a built-in control. The lysate-antibody mixture was incubated in the vessel 119 at room temperature for 5 minutes. A test strip (dipstick) 110 comprising a sample pad, a test area, and an absorption pad is dipped into the lysate-antibody mixture, allowing the aqueous reaction mixture to be wicked up by capillary flow through the sample pad 115 (a cellulose pad), the test area 116, and ultimately the absorption pad 117 (a paper pad). The test area 116, a nitrocellulose membrane, contains a test line 121 and a control line 122 that are pre-coated with appropriate capturing agents (anti-tag antibody and streptavidin, respectively). The test line 121 captures the C4d-anti-C4d complexes formed in the mixture, and the control line 112 captures a built-in control agent to ensure the validity of the test. The colloid gold conjugates yield a vivid red-colored line if captured.

A positive test 131 is indicated by red bands at both the test line 121 and the control line 122. A negative test 132 is indicated by a single colored band at the control line 122. The color intensity of the positive test line correlates with the amount of C4d present in the cell lysate and can be quantified visually or photoimaged and analyzed with software (e.g., Image J). A specific strip reader can also be used for documenting/quantifying the test results.

The capillary flow assays for CFB-CAPs may also be performed using a single anti-C4d antibody in a “competition” format (see FIG. 1B). As described in FIG. 1B, lysates of blood cells 113 are prepared and used for CFB-CAP detection. 9A10E4 anti-C4d antibodies, as both an unlabeled competitor 118 and a tagged capture antibody 111, are added to the cell lysate (depicted as C4d-bound analyte 113 in the drawing). To detect C4d-anti-C4d complexes formed in the cell lysate 113, goat-anti-mouse Ig conjugated with colloid gold 124, along with the gold-labeled biotin control 114, are then added into the mixture. After a 5-minute incubation, a test strip (dipstick) is dipped into the vessel 119 containing the cell lysate-antibody mixture to wicked it up through the strip by capillary flow. The C4d-9A10E4 complexes formed in the lysate are captured at the test line 121. The built-in control agent is captured at the control line 122. A positive test is indicated by both a colored test line 121 and a colored control line 122. A negative test is indicated by a single colored control line 122. The color intensity of the positive test line correlates with the amount of C4d present in the cell lysate and can be quantified visually or with other devices and software.

FIG. 2A shows a method for CFB-CAP detection by capillary flow using a lateral flow assay (LFA) in a multiple test strip format. In this embodiment, capillary flow on multiple nitrocellulose membrane test strips 210A, 210B, 210C, 210D, 210E, and 210F in parallel is utilized. Unlike the dipstick method, a conventional LFA houses the test strip (which we may refer to as 210 for simplicity) in a plastic cassette, and the cassette is placed in a horizontal position during the test. The test strip 210, as illustrated, includes a sample pad 214, a conjugation pad 215, a test area 216, and an absorption pad 217. The sample (blood cell lysate) is applied to the sample pad 214 through a port 219. The sample is wicked by capillary flow force area “laterally” (versus “upwardly” in the dipstick method) through the conjugation pad 215 that has been preloaded with colloid gold-conjugated 9A10E4 anti-C4d or a bifunctional/bispecific derivative mAb, allowing for the detection of CFB-CAPs, such as BC4d, TC4d, EC4d, PC4d, RC4d, and GC4d. A built-in control (gold-conjugated biotin) is also added into the mixture. The lysate-antibody mixture is further wicked through the test area 216 and finally reaches the absorption pad 217. In this design, the test line is coated with goat-anti-mouse IgG to capture the C4d-anti-C4d complexes. As with previous examples, the test area may be formed of a nitrocellulose membrane. A positive test is indicated by both a colored test line 221 and a colored control line 222.

FIG. 2B shows a method for CFB-CAP detection by capillary flow using a lateral flow assay (LFA) with a single strip 250 in a multiplex format. In this embodiment, capillary flow on a nitrocellulose membrane with a single test strip 250 with a test area 256 having multiplex test lines 251A, 251B, 251C, 251D, 251E, and 251F is utilized. Similar to the LFA described in FIG. 2A, a sample (i.e., blood cell lysate) is applied to the sample port 259, and conjugated monoclonal antibody 9A10E4 or a bifunctional/bispecific derivative of the mAb is used to detect the presence of CFB-CAPs such as BC4d, TC4d, EC4d, PC4d, RC4d, and GC4d.

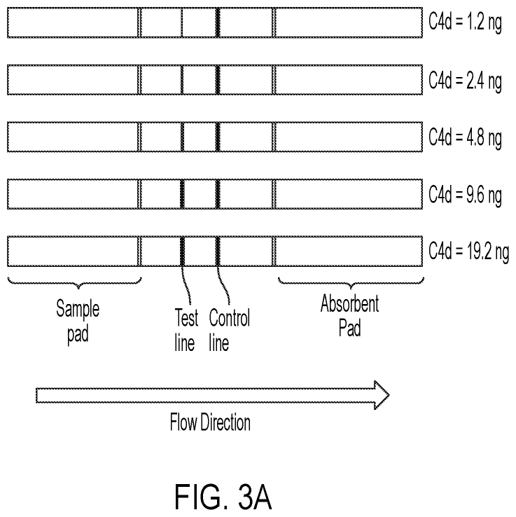

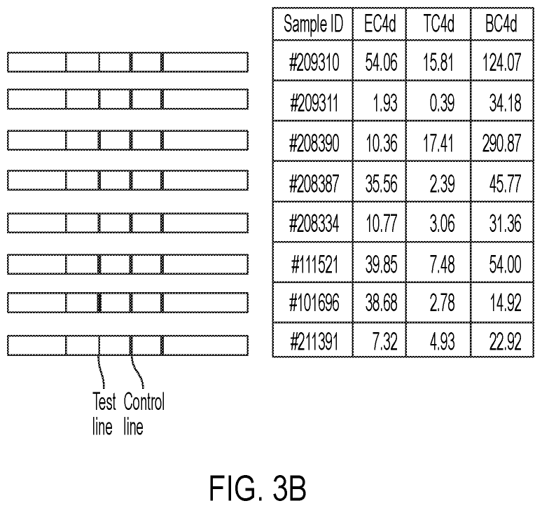

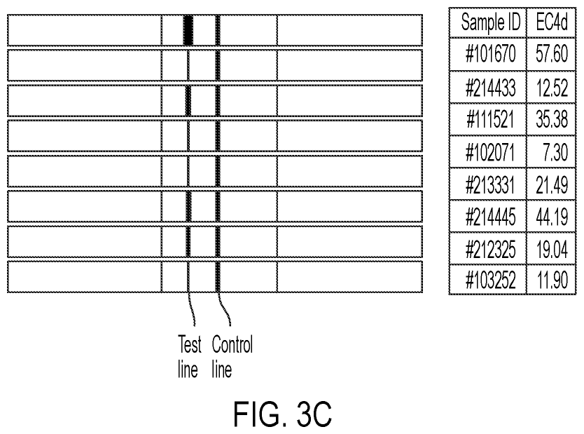

To test the feasibility of capillary flow assays as illustrated in FIG. 1A, experiments were performed using purified C4d at different concentrations as the sample, 9A10E4 conjugated with colloid gold was used as the detection antibody and tagged Quidel anti-C4d was used as the capture antibody (FIG. 3A). The C4d-anti-C4d complexes captured at the test line were visualized by 9A10E4 conjugated with colloid gold. The intensity of the red-colored test line on each test strip correlated with the quantity of C4d detected. This is illustrated by the numeric values in FIG. 3A.

Tests to demonstrate the feasibility of the capillary flow assay for detection of EC4d in red blood cell lysates were also performed using the dipstick method. Lysates of red blood cells prepared from patients with known levels of EC4d as determined by flow cytometry were analyzed by capillary flow assays, as shown in FIG. 1A.

To investigate whether the capillary assays for CFB-CAPs can utilize samples prepared from different types of cells in different preservation conditions, lysates of buffy coat patient samples with known levels of EC4d, TC4, and BC4d as determined by flow cytometry were frozen and then thawed prior to the assay. Cell lysates are prepared and analyzed by capillary flow assay, as shown in FIG. 1A. C4d was detected by mAb 9A10E4 conjugated with colloid gold. As shown by the numeric values in FIG. 3B, The intensity of the colored test lines correlated with the levels of EC4d in each sample.

FIG. 3C shows the feasibility of the capillary flow assay for detection of EC4d in freeze-thawed red blood cell lysates. Lysates of freeze-thawed red blood cell patient samples with known levels of EC4d as determined by flow cytometry were analyzed by a capillary flow assay with detection by 9A10E4.

### Example 2

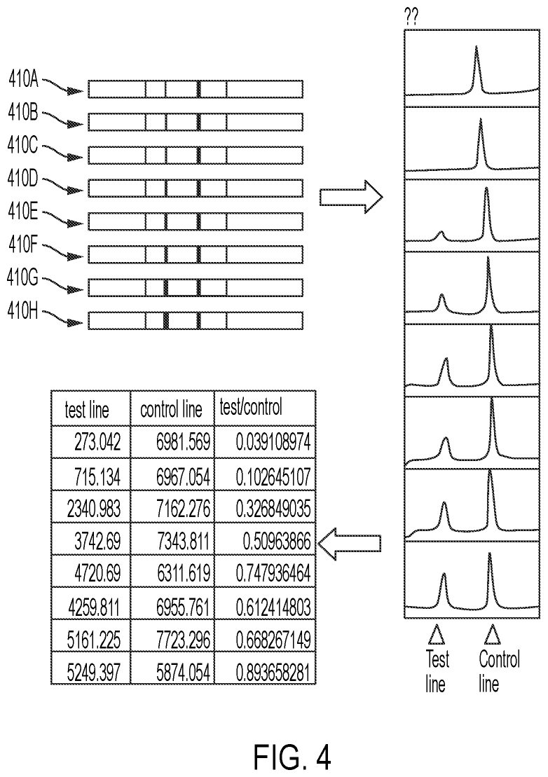

The above test results of capillary assays can be directly visualized and semi-quantitatively analyzed by test line intensities. To achieve precise quantitation, the test results can be photoimaged and digitally analyzed. In this example, red blood cell lysates of patients with known levels of EC4d as determined by flow cytometry were analyzed by capillary flow assay with detection by 9A10E4. The strips were photographed using a digital camera, and the digital file was analyzed using the Image J software (available, for example, from the NIH website). Specifically, the color image was converted into a gray-scale image for further analysis. An area of interest encompassing the test line and control line was identified on each strip. The intensities of the band in the test line and control line were then analyzed using the “Gel Analysis” function in the Image J software. As shown in FIG. 4, the intensities of bands of each test strip 410A, 410B, 410C, 410D, 410E, 410F, 410G, and 410H were illustrated as peaks of different height/width on the right, and the peak areas were quantitated by the software (table at bottom center). The numeric values of the band intensities can be exported to a spreadsheet application or other data file format for further analysis. For example, the intensities of the test lines in individual test strips can be correlated with the EC4d level determined by flow cytometry (correlation graph at bottom left).

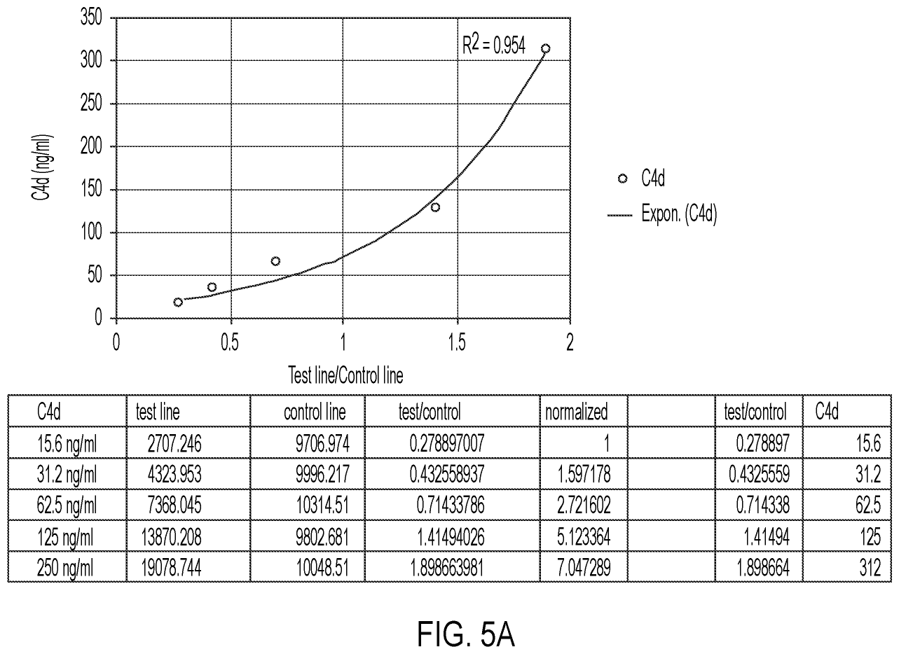

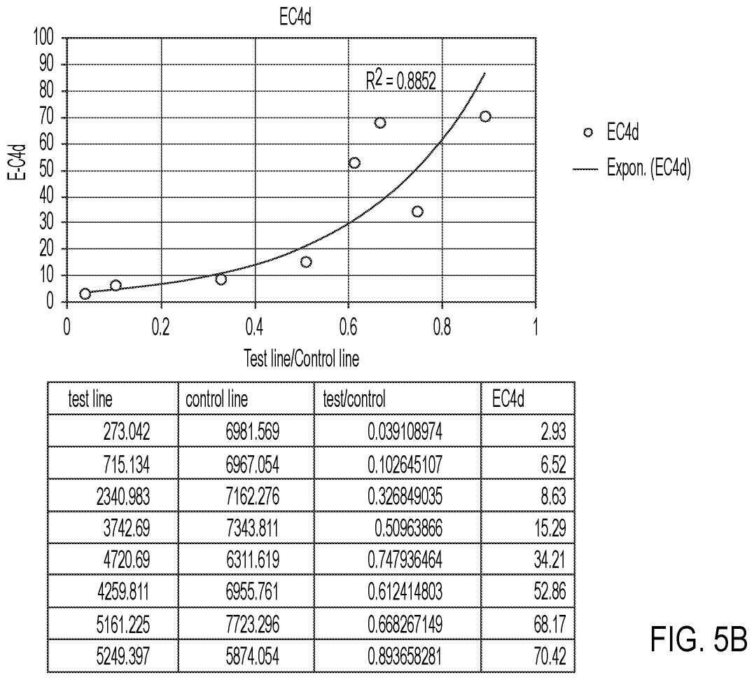

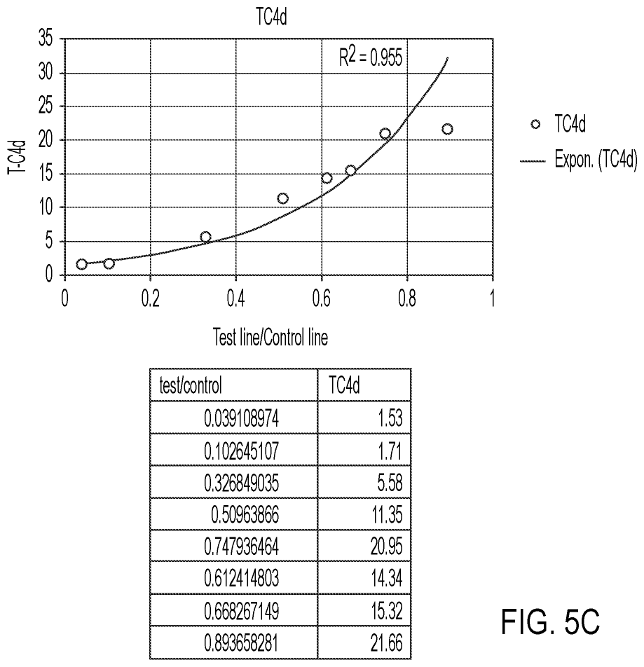

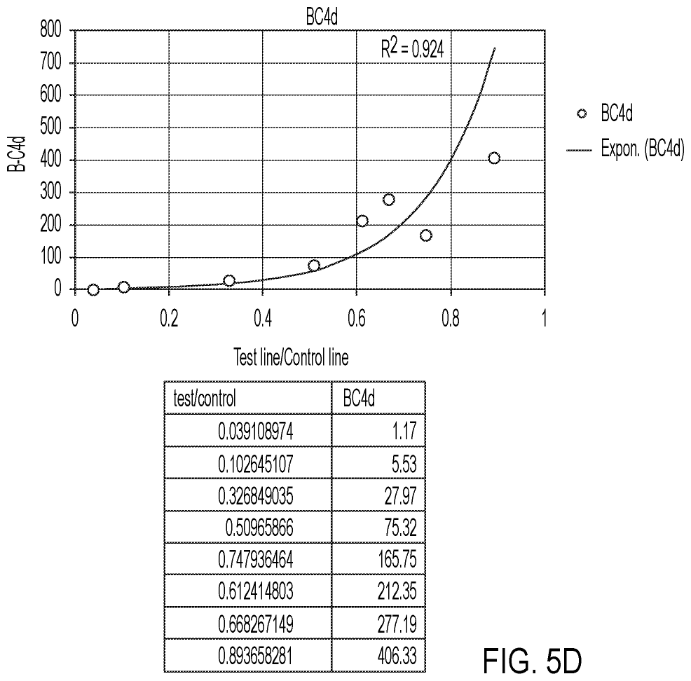

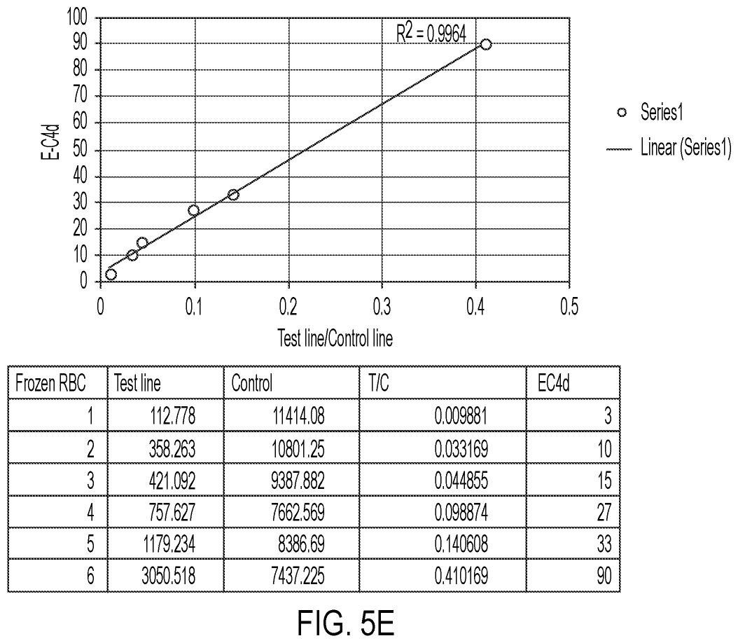

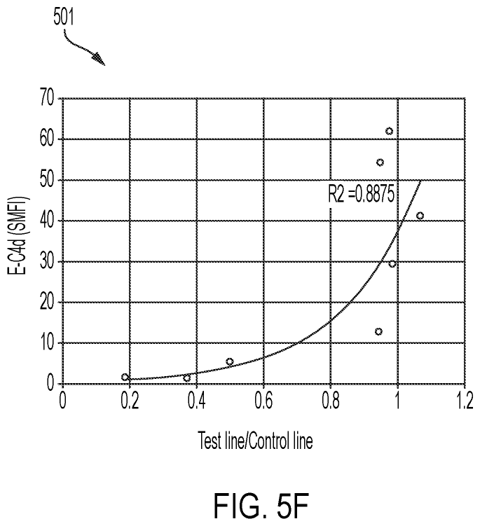

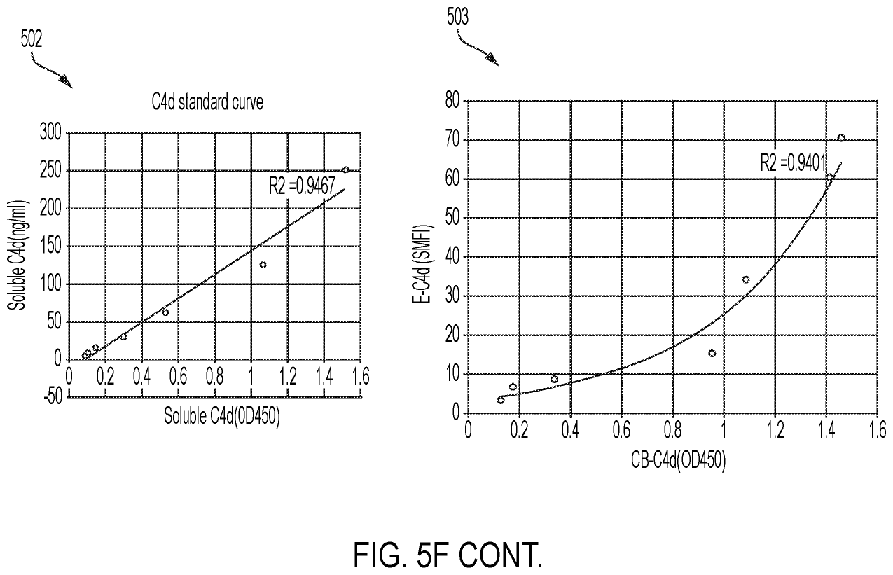

To validate the quantitation method described in FIG. 4, the results of a capillary flow assay with purified C4d were analyzed (see FIG. 3A). FIG. 5A shows a standard curve generated by capillary flow assay of different concentrations of purified C4d. The results demonstrate that the capillary flow assay can quantitatively differentiate C4d at different concentrations. FIG. 5B shows correlation of EC4d levels as determined by flow cytometry versus capillary flow assay. FIG. 5C shows correlation of TC4d levels as determined by flow cytometry versus capillary flow assay. FIG. 5D shows correlation of BC4d levels as determined by flow cytometry versus capillary flow. FIG. 5E shows correlation of EC4d levels in freeze-thawed samples of red blood cells as determined by flow cytometry versus capillary flow assay. FIG. 5F shows a strong correlation of EC4d, BC4d, and TC4d as measured by flow cytometry (cells) 501, ELISA (cell lysates) 502, and LFA (cell lysates) 503.

Using the quantification method described in FIG. 4, the CFB-CAP levels on different cell types measured using different methods were compared. The strong correlations between results of different assays support the validity and utility of these different CFB-CAP measures.

### Example 3

In this example, anti-C4d #1 and anti-C4d #2 are monoclonal antibodies (mAb) that bind respectively to distinct epitopes on the C4d molecule. Several anti-C4d mAb are available commercially, most of which are derived from a limited pool of mAb clones (e.g., clone 10.11, clone 2D11, and clone LP69). Among these antibodies, the anti-C4d mAb available at Quidel Corp. (San Diego, CA; catalog no. A213; clone: 10.11) is one of the most used and cited in the literature. It has also been used in flow cytometric assays for CB-CAPs. Most of these commercial antibodies were generated using C4 purified from human plasma, purified C4d, recombinant C4d, or C4d peptides as the antigen. However, C4d in fluid phase and C4d bound on cell surfaces may be in different conformation and exhibit distinct epitopes. To expand the investigation of CB-CAPs, a new kind of anti-C4d antibodies that are specific to C4d bound to cells will be desirable. Such novel anti-C4d mAb (depicted as Anti-C4d #2) and conventional anti-C4d mAb (depicted as Anti-C4d #1), together, can be utilized in pairs in various immunological assays and allows for development of novel CB-CAP assays. To this end, two mouse anti-C4d mAb (clone 9A10E4 and clone 7G6B1; hereafter referred to 9A10E4 anti-C4d, 7G6B1 anti-C4d, or simply 9A10E4 and 7G6B1) were developed and selected through a standard methodology and identified as unique antibodies that bind to two separate epitopes on C4d. 9A10 binds to an epitope that is distinct from the epitope recognized by the Quidel anti-C4d antibody. 7G6B1 binds to an epitope that is distinct from the epitope recognized by 9A10 but similar to the epitope recognized by the Quidel anti-C4d antibody.

To characterize the binding specificity of these three anti-C4d antibodies (Quidel, 9A10E4 and 7G6B1), competition staining assays were conducted. The results of the anti-C4d competition assay show that Quidel anti-C4d (cat. No. A213) and the new anti-C4d mAb 9A10E4 recognize different epitopes on C4d. RBCs bearing C4d were prepared from a patient with SLE and preincubated with 9A10E4 anti-C4d (competitor) at different concentrations (ranging from 0.5 μg to 10 μg) at 4° C. for 20 min. Quidel anti-C4d (0.2 μg) conjugated with a fluorophore Alexa Fluor 488 (AF488) was then added to stain RBC. After a 20-minute staining period, RBCs were washed and analyzed by flow cytometry. As shown in the histogram, the preincubation with even 200-fold excess of 9A10E4 anti-C4d (10 μg) did not diminish the staining by the Quidel anti-C4d. Similar competition staining assay was conducted with the two new anti-C4d mAb 9A10E4 and 7G6B1. The results show that 9A10E4 and 7G6B1 recognize different epitopes on C4d. The background staining with a mouse IgG1 isotype control is indicated by an arrow. These results demonstrate that these anti-C4d mAb bind to different epitopes on C4d.

To further verify the epitope distinction between the Quidel anti-C4d and 9A10E4 anti-C4d, the competition staining assay was conducted using the Quidel anti-C4d as the competitor and 9A10E4 conjugated with AF488 as the staining antibody. The results of the anti-C4d competition assay show that Quidel anti-C4d and 9A10E4 anti-C4d recognize different epitopes on C4d. As shown in the histogram, the preincubation with an even 200-fold excess of Quidel anti-C4d (10 μg) did not diminish the staining by 9A10E4 anti-C4d. The background staining with a mouse IgG1 isotype control is indicated by an arrow. These results again demonstrate that these two anti-C4d mAb bind to different epitopes on C4d.

It was also found that Quidel anti-C4d and 9A10E4 anti-C4d recognize different epitopes on C4d and generate additive signals when combined. Quidel anti-C4d (conjugated with AF488) and 9A10E4 (conjugated with AF488). The results collaborate with the results of competition staining assays and reinforce that these two antibodies recognize different epitopes on C4d. Therefore, they can be used in pairs in immunoassays that may require two antibodies as the capture antibody and detection antibodies, respectively.

Next, human RBCs were fixed with paraformaldehyde to demonstrate 9A10E4 anti-C4d binds to C4d on fixed cells (indirect staining: 9A10E4 followed by goat-anti-mouse Ig FITC conjugated). The widely used flow cytometry assays for CB-CAPs are limited by the requirement of freshly prepared blood cells in order to maintain C4d recognizable by the anti-C4d mAb used. Anti-C4d mAb that can recognize C4d epitopes on cells preserved by fixatives will undoubtedly broaden the utility of the CB-CAP assays. Therefore, such a potential capacity of 9A10E4 anti-C4d by flow cytometry was investigated. Specifically, C4d-bearing RBC were prepared and fixed or not with different concentration of paraformaldehyde (--- 0%; --- 0.5%; ---1.0%; --- 1.5%; --- 2.0%) at room temperature for 15 min prior to staining with 9A10E4 anti-C4d. The background staining with a mouse IgG1 isotype control is indicated by an arrow. These results demonstrate that 9A10E4 anti-C4d is capable of recognizing C4d on fixed cells.

### Example 4

In this study, monoclonal antibodies with the capacity to individually bind to C4d and a cell type-specific antigen simultaneously were generated. A single monoclonal antibody can bind to C4d and a specific cell type, such as, but not limited to, CD19-B cell, CD3-T cell, or CD42b platelet.

Conventional monoclonal antibodies are generated to each recognize a specific epitope on an antigen. Therefore to recognize two different antigens on the same cell will require two different antibodies, which may sometimes not be feasible for closely located antigens due to steric constraint. Recent advances in molecular biology/recombinant protein technology have allowed for generating recombinant antibodies with specificities for two different antigens (bi-specific antibodies) or with acquired functions (fi-functional antibodies). With the newly generated 9A10E4 antibody (1′ antibody from the left in the bottom row), it was anticipated replacing one of the two antigen-recognizing regions with specificity to, for example, CD19 (a surface molecule expressed on B cells), CD3 (a surface molecule expressed on T cells), or CD42b (a surface molecule expressed on platelets). These recombinant antibodies may function as novel tools to streamline the detection of C4d on B cells, T cells, platelets, etc.

Mouse-human chimeric monoclonal antibodies were generated, with the capacity to bind specifically to C4d or to bind simultaneously to C4d and a cell-type-specific antigen via the Fab mouse domains and to enable the antibody with functional capacity via the human isotype-specific Fc domain. Such functions include but are not limited to Fc receptor binding and complement activation.

Recombinant antibody technology has further allowed the generation of chimeric antibodies composed of different regions derived from two different species. For example, the Fc region of human IgG antibodies contains a binding site for complement protein C1 that confers human antibodies the ability to activate the complement system. Such ability is lacking in mouse IgG1 antibodies. Therefore, 9A10E4 anti-C4d is engineered to generate chimeric antibodies that contain the Fc region-derived human IgG and hence the complement-activating ability. These chimeric antibodies, once available, will enable us to develop novel assays for measuring CFB-CAPs.

### Example 5

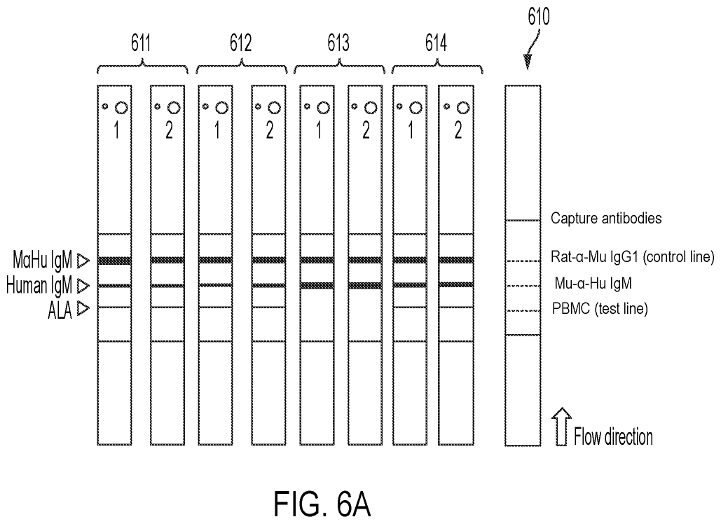

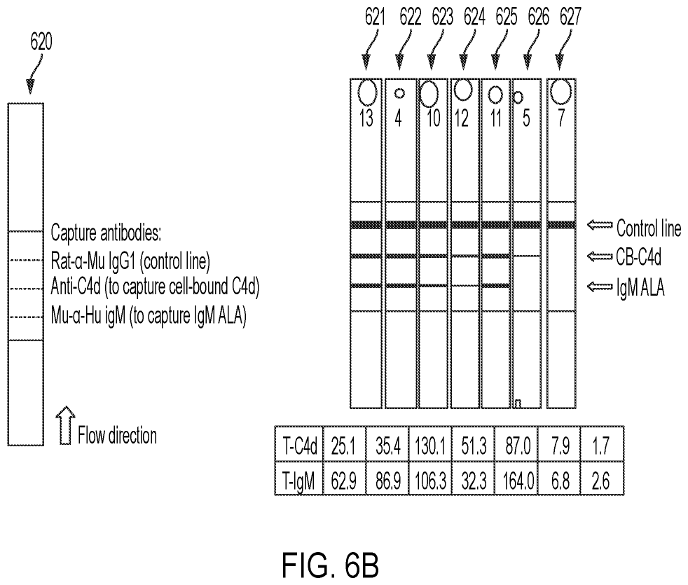

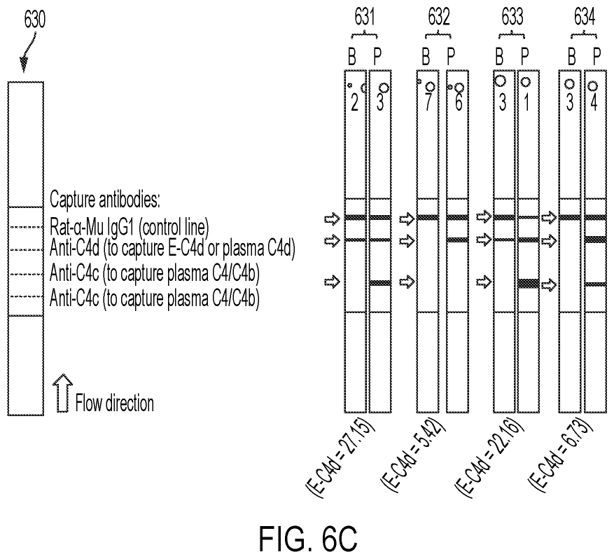

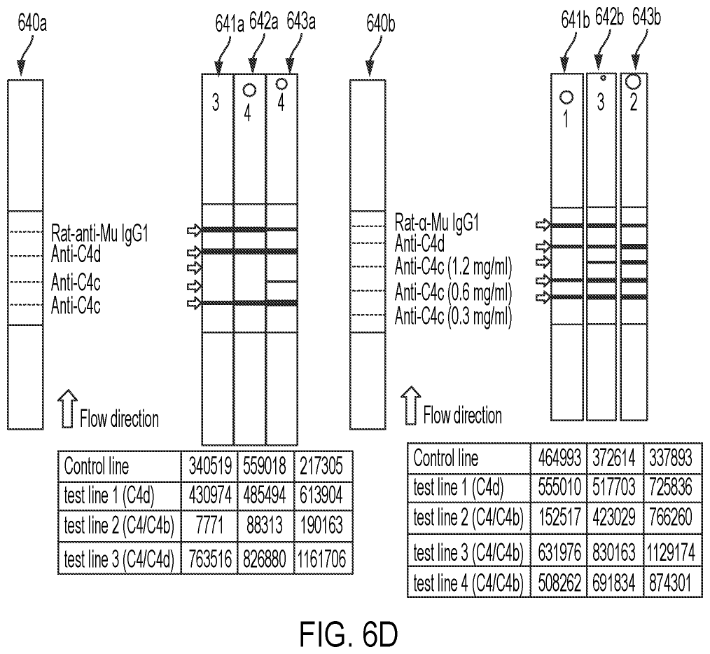

To test the feasibility of a simple detection method for anti-lymphocyte autoantibodies (ALA) present in the plasma of patients with autoimmune diseases, a lateral flow assay (LFA) was designed. The design rationale is outlined as follows (610). By fixing peripheral blood mononuclear cells (PBMC), which consist predominantly of lymphocytes, on an LFA strip as the bait, ALA present in a plasma sample will bind and be captured when the plasma is wicked through the strip. The captured ALA can be visualized by using a mouse-anti-human immunoglobulin M (IgM) monoclonal antibody (mouse IgG1 isotype) conjugated with colloidal gold (shining red color) as a detection antibody. As a consequence, the appearance of a pinkish red band at the position where PBMC was fixed on the strip will indicate the presence of ALA in the test plasma sample (FIG. 6A). It was shown previously that IgM is the most prominent isotype of ALA. Therefore, this assay was focused on detecting IgM ALA. As controls for assay validity, two monoclonal antibodies were also fixed on the LFA strip. The first is another mouse-anti-human IgM (Mu-α-Hu IgM) which was used to demonstrating the ability for capturing non-ALA IgM in the plasma sample. The second, a rat-anti-mouse IgG1 (Rat-α-Mu IgG1), serves as the conventional control for ensuring the quality of gold conjugation as well as the antigen-antibody binding function. The assay is considered valid when both control bands are positive. The configuration of a 6-cm long LFA strip, with a 2.5 cm-long nitrocellulose membrane reaction area franked by bottom- and top-absorption pads, is illustrated on the right. In the pilot study, the LFA strips were used in a dip-stick manner (upright flow). A schematic illustration of the LFA strip is shown on the right.

PBMC was isolated from healthy individuals by Ficoll gradient centrifugation and resuspended in PBS at approximately 2×10⁹ cells/ml. One to 1.5 μl of PBMC suspension was carefully deposited on an LFA strip using a dip pen. Mu-α-Hu IgM (Invitrogen) and Rat-α-Mu IgG1 (Invitrogen) (both at 1 mg/ml) were similarly deposited on the strip. The strips were then dried at 37° C. for 1 hour and stored at 4° C. until use. At the time of the assay, 5 μl of patient plasma was diluted to 100 μl with a reaction buffer (Tris-buffered saline (TBS) containing 1% Tween-20) in a microwell of a 96-well plate. A pre-prepared LFA strip was then placed into the microwell containing the diluted plasma sample and maintained in the upright position for 30 minutes until the plasma sample was completely wicked up. This step allows the ALA potentially present in the plasma to be captured and retained by PBMC fixed on the LFA strip. Subsequently, mouse-anti-human IgM mAb (BD Biosciences) conjugated with colloidal gold (using Gold conjugation kit from Abcam) was diluted in 100 μl of reaction buffer, added to the same microwell, and allowed to be wicked up through the LFA strip. This second step allows ALA retained by PBMC to bind gold-conjugated mouse-anti-human IgM and become visible as a pinkish red band on the strip. Similarly, the control bands would appear as pinkish red bands at respective positions on the LFA strip. In general, the reaction bands would begin to appear within 10 minutes and reach maximal/stable intensity in approximately 40 minutes.

Each pair of LFA strips with fixed PBMC isolated from two individuals were tested with the diluted plasma of a respective lupus patient. As shown in the image on the left, positive pinkish red bands were visible on strips tested with plasma of patients #107395 (611), #128674 (612), and #214507 (614), but not with plasma of patient #214328 (613). These results suggest the presence of ALA in 3 of the 4 plasma samples tested. The results of this experiment support the feasibility of detecting ALA in a patient's plasma using a simple LFA assay (FIG. 6A).

### Example 6

Both complement activation products (e.g., C4d) and ALA are presented, concurrently or alone, on the surface of T lymphocytes in a fraction of lupus patients. It was therefore hypothesized that C4d and ALA associated with lymphocyte membranes can be “dissolved” when patient's lymphocytes are lysed; such C4d and ALA present in the lysate can react with anti-C4d and anti-immunoglobulin antibodies and can be detected using lateral flow assay. To test this hypothesis, LFA strips coated with both anti-human IgM mAb and anti-C4d mAb (“capture antibodies”) were prepared (620). The test sample (cell lysate that contains cell fragments) was first incubated with colloidal gold-conjugated anti-human IgM and anti-C4d antibodies (“detection antibodies,” which recognize antigenic epitopes different from those of capture antibodies). The lysate-antibodies mixture was then run through the LFA strip. CFB-C4d and/or ALA (complexed with detection antibodies) present in the sample will be captured by capture antibodies on the strip and visualized as pinkish red bands at indicated positions. As a control for assay validity, a rat-anti-mouse IgG1 (Rat-α-Mu IgG1) was fixed on the strip for ensuring the quality of gold conjugation as well as the antigen-antibody binding function. The configuration of the LFA strip is illustrated on the left.

PBMC of patients with SLE or other autoimmune diseases were isolated using Ficoll gradient centrifugation and lysed with phosphate-buffer saline (PBS) containing 0.5% Triton X-100 (at 2×10⁹ cells/ml). The lysate was centrifuged to remove insoluble residues (nuclei, etc.) and stored at −20° C. until use. LFA strips coated with capture antibodies were prepared as described above. At the time of the assay, 10 μl of cell lysate was diluted with 90 μl of reaction buffer (TBS/1% Tween-20) in a microwell and incubated for 10 min with colloidal gold-conjugated mouse-anti-human IgM mAb and gold-conjugated mouse-anti-human C4d mAb. A pre-coated LFA strip was then placed into the microwell containing the lysate-mAb mixture and kept in an upright position until the reaction mixture was completely wicked up through the strip. In general, the reaction bands would begin to appear within 10 minutes and reach maximal/stable intensity in approximately 40 minutes.

In assays using PBMC lysates (containing cell fragments) prepared from 5 patients with SLE (#102357 (621), #157292 (622), #209327 (623), #214507 (624), and #214520 (625); see photograph on the right), pinkish red bands were visible at the positions where Mu-α-Hu IgM and anti-C4d capture antibodies were located. In contrast, only faint bands or no band were visible when PBMC lysates of two patients (626 and 627) with other autoimmune diseases were tested. A positive control band was present in all tests, indicating the validity of the assay. Moreover, the intensity of the pinkish red bands correlates with the levels of surface-bound C4d and IgM ALA on T cells measured by flow cytometry (T-C4d and T-IgM, respectively). The results of this study support the feasibility of a simple duplexed LFA assay for detection of CFB-CAP and ALA simultaneously (FIG. 6B).

### Example 7

Major advantages of lateral flow assays, such as their simplicity in assay technology, short assay time, and requirement for small sample size, make them ideal candidates as point-of-care (PoC) tests. PoC tests may provide important information and facilitate the identification of patients for next-tier tests and/or timely diagnosis/treatment. Therefore, the development of a PoC test for quick identification of patients with elevated levels of erythrocyte-bound C4d (E-C4d) and/or abnormal C4/C4b/C4d levels in the plasma was explored. This test will alert physicians to a potential diagnosis of SLE and prompt further test/treatment decisions. Mouse mAb reactive with an epitope within human C4c (anti-C4c) and human C4d was deposited on an LFA strip as the capture antibodies (630). The antigenic reactivity of anti-C4c is restrictive to C4 and C4b, and thus serves as the capture of C4 in the plasma. Two anti-C4c capture antibodies are sequentially placed on the LFA strip to ensure complement capture of C4, which is present at high levels in the plasma, and also serve as a comparative measure of plasma C4 levels. Patient samples with increased or high normal levels of plasma C4 are expected to yield two C4 bands. Patient's blood will be collected, separated, and processed to generate a pair of test samples (“whole blood cell fragment-containing lysate” and “diluted plasma). The test samples will be diluted with appropriate reaction buffers and incubated with anti-C4d detection antibody (recognizing an epitope present in C4, C4b, and C4d) conjugated with colloidal gold. The test sample-antibody mixture will then be run through the LFA strip. The presence and levels of CFB-C4d and plasma C4/C4b/C4d can be visualized by the appearance of pinkish red bands on the LFA strip. Because the whole blood contains predominantly erythrocytes, the CFB-C4d detected in the whole blood lysate represents primarily erythrocyte-bound C4d. A schematic illustration of the assay is shown on the left.

LFA strips with capture antibodies were prepared as described above and stored at 4° C. until use. Patient blood samples can be collected by either finger prick or conventional method. One μl of the whole blood was placed into a microtube containing 1 ml of PBS and centrifuged for 30 seconds in a microcentrifuge. The supernatant was transferred into a fresh tube and saved as the “diluted plasma” sample. The blood cell cells were again diluted with 1 ml of PBS and centrifuged for 30 seconds. The PBS was removed, and the resulting cell pellet was lysed with 10 μl of lysis buffer (PBS/0.5% Triton X-100) (“whole blood lysate”). The whole blood CF-containing lysate was diluted with a reaction buffer (PBS/1% Tween-20) to 100 μl in a microwell and incubated with colloidal gold-conjugated anti-C4d. Fifty μl of the diluted plasma was supplemented with 50 μl of PBS/2% Tween-20 and incubated with colloidal gold-conjugated anti-C4d in another microwell. After a 10-minute incubation at room temperature, pre-prepared LFA strips were placed into the microwells and allowed for the test samples to be wicked up. In general, the reaction bands would begin to appear within 10 minutes and reach maximal/stable intensity in approximately 40 minutes.

Paired whole blood cell fragment-containing lysate (B) and diluted plasma (P) of 4 lupus patients were tested (FIG. 6C). E-C4d bands were visible in two patient samples (#111521 (631) and #208375 (632)). In the plasma samples of these two patients, strong C4 bands and weak C4d bands were visible. Noted also was that a second weak C4 band was visible in the plasma sample of #208375 (633), suggesting a higher plasma C4 level. Together, these patterns suggest that both patients had elevated CFB-C4d levels (representative of E-C4d levels) and relatively normal plasma C4/C4d levels (compared to normal samples not shown here), indicative of complement activation on blood cell surfaces.

In contrast, no CFB-C4d band was visible in patient samples #214512 (632) and #214520 (634), suggesting low E-C4d levels. In the plasma sample of #214512 (632), a strong C4d band, but no C4 band, was visible. In the plasma sample of #214520 (634), a C4d band with increased intensity and a C4 band with decreased intensity were visible in the plasma sample of #214520 (634). These latter results indicate that both #214512 (632) and #214520 (634) had normal E-C4d but abnormally decreased plasma C4 and elevated plasma C4d levels, indicative of increased complement activation in the fluid phase. The results of this study support the feasibility of a simple and rapid LFA assay for detecting complement activation, present as cell-bound or free circulating in plasma, in a patient (FIG. 6C).

### Example 8

The above-described LFA for detecting CFB-C4d (E-C4d) and plasma C4/C4b/C4d shows the potential as a useful PoC test for rapid and qualitative screening of patient samples. Next, an LFA that would allow for (semi)quantitative measure of plasma C4 was designed. Anti-C4c mAb at different concentrations were deposited in sequence on an LFA strip (640a and 640b). It is anticipated that a gradient of binding patterns (number of bands, intensity of bands) will be generated in proportion to the levels of C4 in the tested plasma samples. Ultimately, a “reference” pattern can be generated using plasma samples with known C4 levels and used to derive C4 levels in test samples.

Diluted plasma samples of 3 lupus patients were prepared and tested using the LFA strips as described above in FIG. 6C. The C4 and C4d bands of different intensities were visible in the 3 samples, indicating different plasma C4 and C4d levels (FIG. 6D) (641a, 641b, 642a, 642b, 643a, and 643b). The band intensities on the LFA strips were also analyzed using an LFA reader (Model: RDS-2500; Detekt) and shown below the photograph. The results showed that patient #102359 (641a and 641b) had the lowest plasma C4 and #208375 (643a and 643b) the highest plasma C4. Plasma samples of the same patients were tested on the newly designed (semi)quantitative LFA strips. As expected, the sample of patient #102359 (641a and 641b) yielded two weaker C4 bands compared to the 3 stronger C4 bands in the other two patient samples. Again, patient #208475 (643a and 643b) appeared to have the highest plasma C4 levels, as shown by the 3 most prominent C4 bands.

1. A method of determining a level of a complement activation product in a patient, comprising:
contacting a sample comprising one or more analytes with a detection agent, wherein the one or more analytes comprise a cell fragment and a complement activation product, wherein the complement activation product is attached to the cell fragment (CFB-CAP), and wherein the detection agent comprises a detection antibody that specifically binds to the CFB-CAP and facilitates detection of the CFB-CAP in at least one of the one or more analytes;
immobilizing the at least one of the one or more analytes in a fluid path; and
determining a level of the CFB-CAP in the at least one of the one or more analytes at one or more locations in the fluid path by determining a level of binding of the detection antibody to the CFB-CAP.

2. The method of claim 1, wherein the CFB-CAP is attached to at least a fragment of erythrocytes, lymphocytes, reticulocytes, platelets, granulocytes, monocytes, eosinophils, or basophils.

3. The method of claim 1, wherein: the sample comprises at least one entity selected from erythrocytes, lymphocytes, reticulocytes, platelets, granulocytes, monocytes, eosinophils, and basophils; and the method comprises lysing the at least one entity in the sample before contacting the sample with the detection agent.

4. (canceled)

5. The method of claim 1, wherein the sample comprises a blood sample.

6. The method of claim 1, wherein the sample, after being obtained from the patient, has been left at a temperature equal to or above 4 degrees Celsius for at least a period of time before being contacted with the detection agent.

7. The method of claim 3, wherein at least a portion of the entity has been spontaneously lysed.

8. The method of claim 6, wherein the period of time is at least about 60 minutes.

9. The method of claim 1, wherein the CFB-CAP comprises a cell fragment-bound C4d.

10. The method of claim 9, wherein the detection antibody comprises an anti-C4d antibody

11. The method of claim 9, wherein the cell fragment-bound C4d is a complement activation product selected from BC4d, TC4d, EC4d, PC4d, RC4d, GC4d, MC4d, and combinations thereof.

12. The method of claim 1, wherein the at least one of the one or more analytes is immobilized in the fluid path by a capture antibody.

13. The method of claim 12, wherein the capture antibody binds to a different epitope on the CFB-CAP from one to which the detection antibody binds or binds to the detection antibody.

14. (canceled)

15. (canceled)

16. The method of claim 12, wherein the one or more analytes further comprise an anti-T cell antibody, a freely circulating complement activation product (FC-CAP), or a combination thereof.

17. The method of claim 16, comprising determining a level of at least one of the CFB-CAP, the anti-T cell antibody, and the FC-CAP in the one or more analytes.

18. (canceled)

19. The method of claim 16, wherein the capture antibody comprises a first capture antibody that binds to the CFB-CAP and a second capture antibody that binds to the anti-T cell antibody or the FC-CAP, and wherein the first capture antibody and the second capture antibody are configured to immobilize the CFB-CAP and the anti-T cell antibody or the FC-CAP at least two separate locations in the fluid path.

20. The method of claim 16, comprising determining a level of each of (a) the CFB-CAP and (b) the anti-T cell antibody or the FC-CAP in a single fluid path.

21. The method of claim 16, wherein the anti-T cell antibody is an anti-T cell autoantibody.

22. (canceled)

23. (canceled)

24. (canceled)

25. (canceled)

26. (canceled)

27. (canceled)

28. (canceled)

29. The method of claim 16, wherein the capture antibody comprises a first capture antibody that binds to the CFB-CAP, a second capture antibody that binds to the FC-CAP, and a third capture antibody that binds to the anti-T cell antibody, wherein the first capture antibody, the second capture antibody, and the third capture antibody are configured to immobilize the CFB-CAP, the FC-CAP, and the anti-T cell antibody at two or more separate locations in the fluid path or in a single fluid path.

30-35. (canceled)

31. (canceled)

32. (canceled)

33. (canceled)

34. (canceled)

35. (canceled)

36. The method of claim 12, wherein the detection antibody or the capture antibody is the bispecific antibody comprising a first antigen-binding arm binding to C4d and a second antigen-binding arm binding to any one of CD3, CD4, CD5, CD8, CD45, CD19, CD20, CD21, CD22, CD23, CD25, CD40, CD42b, CD69, CD70, CD79, CD80, CD85, CD86, CD137, CD138, CD252, and CD268.

37. (canceled)

38. (canceled)

39. The method of claim 1, wherein the step of immobilizing is performed before the step of contacting.

40. A method of identifying lupus or pre-lupus in a patient, comprising:
obtaining a blood sample from the patient;
determining a level of the CFB-CAP in the blood sample by the method of claim 1;
comparing the determined level of the CFB-CAP with a control level and determining whether the determined level is elevated as compared to the control level; and
determining that the patient has lupus or an increased risk of developing lupus if the determined level of the CFB-CAP is elevated as compared to the control level.

41. (canceled)

42. (canceled)

43. (canceled)

44. (canceled)

45. (canceled)

46. (canceled)
